(19) United States
(12) Patent Application Publication
(10) Pub. No.: US 2005/0074485 A1
(43) Pub. Date: Apr. 7, 2005

(54) Anti-inflammatory/anti-microbial peptides for use in dialysis

(76) Inventor: James M. Lipton, Woodland Hills, CA (US)

Correspondence Address: PERKINS COIE LLP, POST OFFICE BOX 1208, SEATTLE, WA, 98111-1208, US

(21) Appl. No.: 10/845,778

(22) Filed: May 14, 2004

Related U.S. Application Data
(60) Provisional application No. 60/470,606, filed on May 14, 2003.

(51) Int. Cl. A61K 38/10; A61K 38/08; A61K 38/06; A61L 15/00

(52) U.S. Cl. 424/445; 514/2; 514/13; 514/14; 514/15; 514/16; 514/17; 514/18

(57) ABSTRACT

A composition and method of treatment are disclosed in which anti-microbial and anti-inflammatory peptides are used for hemodialysis and peritoneal dialysis in dialysate and gel. An unexpected result of the invention has also been disclosed wherein the anti-microbial and anti-inflammatory peptides may be used in peritoneal dialysis to increase diuresis. Other embodiments of the invention include use of the anti-microbial and anti-inflammatory peptides in locking solutions used for catheters and other vascular access tubing or peritoneal access tubing.

### PRIORITY CLAIM

This application claims priority to U.S. Provisional Application Ser. No. 60/470,606, filed May 14, 2003, which is incorporated by reference as if fully set forth herein, including drawings.

### FIELD OF INVENTION

The current invention relates to the field of treatment and prevention of infection, inflammation and increasing diuresis in dialysis.

### BACKGROUND OF THE INVENTION

Before dialysis became available, total kidney failure was a terminal illness. Today, people with kidney failure can live because of treatments such as dialysis and kidney transplant often allow individuals afflicted with kidney failure an opportunity to live. Dialysis is a method of cleansing an individual's blood when the kidneys fail to function properly. Dialysis eliminates the body's wastes, extra salt and water, and helps to control the blood pressure.

There are two types of dialysis, hemodialysis and peritoneal dialysis. In hemodialysis, a patient is connected via tubes to a kidney machine. Blood is pumped out of the body to the machine, which filters the blood, and then the blood is returned to the body. In peritoneal dialysis, the inside lining of the patient's peritoneal cavity is used as a natural filter. Wastes are removed by means of a fluid called dialysate, which is washed in and out of the peritoneal cavity in cycles. These cycles are called exchanges.

For peritoneal dialysis, a catheter is surgically placed in the peritoneal cavity. A sterile dialysate is delivered into the peritoneal cavity through the catheter. After the filtering process is finished, the dialysate fluid is removed from the body through the catheter. Catheters are also commonly used in hemodialysis and placement is at or about the neck.

The main types of peritoneal dialysis include: Continuous Ambulatory Peritoneal Dialysis (CAPD); and Continuous Cycling Peritoneal Dialysis (CCPD). The basic treatment is the same for each. However, the number of treatments and the way the treatments are performed make each treatment unique.

CAPD is “continuous,” machine-free and accomplished at some point during a patient's normal activities of daily living such as at work or school. A patient accomplishes the treatment by placing a prescribed amount of dialysate, which is supplied in a plastic bag, into the peritoneal cavity and draining the dialysate from the peritoneal cavity a prescribed number of hours later. This is done by connecting a sterile container of dialysate to a catheter in the peritoneal cavity. Raising the plastic bag to shoulder level causes gravity to draw the fluid into the peritoneal cavity. When empty, the container, which held the dialysate, is removed and discarded. When an exchange (putting in and taking out the fluid) is finished, the fluid (which now contains wastes removed from the blood) is drained from the peritoneal cavity and discarded. This process usually is performed three, four or five times in a 24-hour period while a patient is awake. Each exchange takes about 30 to 40 minutes.

CCPD differs from CAPD in that a machine (cycler) delivers and then drains the dialysate for a patient. The treatment usually is performed at night while a patient sleeps. Thus, the system is automated.

Hemodialysis and peritoneal dialysis have been performed since the mid 1940's. Dialysis, as a regular treatment, was begun in about 1960 and is now a standard treatment all around the world. CAPD began in about 1976. Thousands of patients have been helped by these treatments. Many medical practitioners maintain that CAPD and CCPD have several benefits when compared to hemodialysis and as many medical practitioners maintain that hemodialysis is superior to CAPD or CCPD. With continuous dialysis, a patient can control extra fluid more easily, and this may reduce stress on the heart and blood vessels. Additionally, with CCPD a patient is able to eat more and use fewer medications as well as being able to perform more of the activities of daily living such as work or travel.

Another type of peritoneal dialysis used in the United States is called Intermittent Peritoneal Dialysis (IPD). IPD is usually performed in the hospital due to the repeated short exchanges and dietary control. Most people in need of IPD need about 36-44 hours of IPD weekly, resulting in two or three days away from home.

In IPD, the dialysis fluid is left in the peritoneal cavity for a short period of time and then is drained out. Again, one complete cycle is called an exchange, and an IPD exchange takes about one hour to complete. In intermittent peritoneal dialysis, diet and fluid intake are usually restricted to control the build up of food wastes and water. In contrast, in continuous peritoneal dialysis, a patient is allowed a more liberal diet.

Some types of acute kidney failure resolve to some functional state after treatment. In some cases of acute kidney failure, dialysis may only be needed for a short time, until the kidneys function within normal limits. In chronic or end stage kidney failure, the kidneys do not return to functionality and dialysis is required. If a patient is a suitable candidate, a patient may be placed on a waiting list for a new kidney. However, very few suitable candidates match with potential kidney donors, which continues to put dialysis as the first line treatment for end stage renal failure.

Dialysis can be performed in a hospital, in a dialysis unit that is not part of a hospital, or at home. In hemodialysis, an artificial kidney (hemodialyzer) is used to remove waste, extra chemicals and excess fluid from the blood. To transfer a dialysis patient's blood into the artificial kidney, vascular access is required. This is usually accomplished by minor surgery to the arm or leg wherein access to the dialysis patient's blood stream is achieved. For example, a common access is made by joining an artery to a vein under the skin and thus creating a fistula. The fistula is then penetrated, allowing access to blood. If the blood vessels are not adequate for a fistula, a graft may be used to join an artery and a vein under the skin. Occasionally, an access is made by means of a catheter, which is inserted into a large vein in the neck. This type of access may be temporary, but in many instances may be used for long-term treatment. As with any vascular access or catheter treatment, the catheter creates a direct communication between a patient's aseptic internal environment and the outside world. The common complications of this communication are infections, inflammations and thrombotic or infiltrative blockage of the access.

Infections and blockage, as well as inflammation, represent only a few of the many possible medical complications associated with dialysis. In fact, although the benefits of hemodialysis and peritoneal dialysis are numerous, many of the complications associated with each may be life threatening. Each technique has its own or similar complications including, but not limited to, ultrafiltration failure, compliance, obesity, clearance, and poor fluid compliance. (Wilkie, M E, et al., Perit Dial Int, 17:87-88 (1997).)

Infection is a problem common to all techniques of dialysis. However, each technique may manifest an infection in a different way common to that technique. Peritonitis, for example, is an infection concern in peritoneal dialysis, whereas graft infection is an infection concern in hemodialysis. Wound and catheter infections are also common in hemodialysis. On the average, peritonitis, although decreased in recent years, presents at least once in every 24 months of treatment per patient. (Id.) It is at least one object of the present invention to address these infection concerns and specifically, peritonitis.

Statistics and reports on infection vary across the United States and the rest of the world. Clearly, however, infection is a major cause of morbidity and mortality among patents with end stage renal disease (ESRD). The United States Renal Data System records indicate that infection is the second leading cause of death among patients with ESRD, following only cardiovascular disease. (United States Renal Data System (1998) Annual Data Report National Institute of Health, Diabetes and Kidney Disease, Bethesda, Md.) Sepsis accounts for 75% of these infection deaths. In one study, almost 16% of deaths in peritoneal dialysis (PD) patients were due to peritonitis, inflammation of the peritoneal space resulting from microbial invasion. Peritonitis and catheter-related infections remain the two most common causes of PD failure. (Furth S I, Donaldson La, Sullivan E K, Watkins S L Peritoneal dialysis catheter infections and peritonitis in children: NAPRTCS Report Pediatr Nephrol (2000) 15:175-82. Okechukwu, C N, Schwartz R D Peritoneal dialysis-associated peritonitis Current Treatment Options in Infectious Disease. Biomed Central (2004) 1-22.). The costs in suffering and in medical care are marked. In the US, septicemia requiring hospitalization has been a consistent association in PD patients for years.

Staphylococcus aureus represent about 60 percent of the isolated bacteremic identification episodes in hemodialysis patients. It represents 30 percent of catheter line sepsis episodes of all types of patients. It represents about 25 percent of peritonitis episodes and nearly 80 percent of the flesh infections associated with peritoneal dialysis. These flesh infections are exit site or “tunnel infections” associated with the indwelling catheter used in peritoneal dialysis. (Golper, T. A. Multidrug Resistant Bacteria (VRE and MSRA) in the Hemodialysis and Peritoneal Dialysis Patient. (Presented at the Seventh Annual Spring Clinical Nephrology Meetings, National Kidney Foundation.) <http://www.hdcn.com/symp/98nkf/golp/gol1.htm>.) S. aureus related peritonitis can be mild to severe. Severe infections may lead to a change of modality in dialysis from peritoneal dialysis to hemodialysis.

Enterococcus sp. also play a significant role in dialysis infections. Many of these infections, 5 percent and growing, are Vancomycin Resistant Enterococcus (VRE), and are severely problematic. VRE, like Methicillin Resistant Staphylococcus aureus (MSRA) have developed resistance to many first line antibiotic medications. One common treatment for MSRA was vancomycin, but like many strains of bacteria, resistance developed.

In the late 1980's and by 1993, the Ad Hoc Committee for the Treatment of Peritonitis had recommended vancomycin as empiric therapy for peritonitis treatment. In time, the empiric use of vancomycin for peritonitis created a type of Enterococcus infection of the peritoneum which was resistant to most commonly used antibiotics. Resistant Enterococcus infections, in most cases, proved fatal to the patient. (Golper T. A. Multidrug Resistant Bacteria (VRE and MSRA) in the Hemodialysis and Peritoneal Dialysis Patient. Presented at the Seventh Annual Spring Clinical Nephrology Meetings, National Kidney Foundation. <http://www.hdcn.com/symp/98nkf/golp/gol1.htm>.) New medications are currently being tested but as many as 85 percent of Enterococcus infections are due to Enterococcus faecalis, which has continued to be resistant to antibiotic treatment. Moreover, reported cases of Vancomycin Resistant Staphylococcus aureus (VRSA) have been reported. (Id. at <http://www.hdnc.com/symp/98nkf/golp/gol2.htm>.) Medications needed to treat these resistant infections are commonly neurotoxic and damage internal organs, and are limited in their killing ability. A preventive method and treatment for these infections is needed that will safely eliminate infection while avoiding serious side effects. Further, a medication is needed that will not interrupt treatment nor require a modality shift. A suitable treatment may exist in a combination of medications for use with the dialysate, locking solutions and wound care.

It is at least one object of the invention to decrease the incidence of peritonitis in peritoneal dialysis by using a dialysate composition containing anti-inflammatory and anti-microbial peptides. It is a further object of the invention to create a composition for use in catheter care, wound care and as a locking solution.

### SUMMARY OF THE INVENTION

The current invention relates to the field of infection and inflammation treatment, control and prevention in dialysis. Experiments associated with the development of this invention also revealed the unexpected result that the invention increased diuresis. Compositions for use in the treatment, prevention and control of infection are disclosed herein using anti-microbial and anti-inflammatory peptides which contain Lysine-Proline-Valine (KPV) at their C-terminus. Preferred peptides are those in which the length of the peptide iw between 3 and 13 amino acids. One preferred peptide is KPV (SEQ ID NO: 1 ). Another preferred peptide that contains KPV at its C-terminus is a dimer of CKPV (SEQ ID NO: 4).

Tests on animals with addition of VPKC-s-s-CKPV (SEQ. ID NO. 4) (hereon referred to as the “KPV dimer”), to peritoneal fluid were without negative side effects supporting the use of this active peptide additive to treatments involving the peritoneal cavity.

Repeated administration of a gel containing the KPV dimer (SEQ ID NO: 4) to peritoneal catheter exit sites in rabbits that had been inoculated with Staphylococcus epidermidis or Staphylococcus aureus either killed completely or reduced the bacteria to the point where it was not possible to culture them from swabs taken from skin sites. Repeated tests on peritoneal catheters inoculated with these two opportunistic bacteria, known to be common on human skin, showed that the KPV dimer (SEQ ID NO: 4) applied in saline locking solution killed these bacteria in vitro. Similar results were found when intraperitoneal catheters were inoculated with the same species of bacteria and the KPV dimer (SEQ ID NO: 4) was applied in the same locking solution in live rabbits.

The findings disclosed herein, in terms of in vitro antimicrobial data and studies in animal models of peritoneal dialysis, offer substantial evidence of the usefulness of the KPV dimer (SEQ ID NO: 4) and other peptides disclosed herein as an adjunct or main line treatment in control of infection and inflammation in dialysis patients.

In one aspect of the invention, anti-microbial and anti-inflammatory peptides containing KPV at the C-terminus are included in a therapeutic amount in a dialysate for use in treatment and prevention of peritonitis. For this application, the term “anti-microbial and anti-inflammatory peptides” is understood to mean peptides and polypeptides that contain KPV at the C-terminus and have anti-microbial and anti-inflammatory properties. Preferred anti-microbial and anti-inflammatory peptides are 3-13 amino acids in length and may be selected from the group consisting of KPV (SEQ ID NO: 1), HFRWGKPV (SEQ ID NO: 2), HdNIeRWGKPV (SEQ ID NO: 3) the KPV dimer (SEQ ID NO: 4), SYSMEHdFRWGKPV (SEQ ID NO: 5) (hereon referred to as “Phe7”), α-MSH (SEQ ID NO: 6) and [Nle₄ dPhe₇]α-MSH (SEQ ID NO: 7) (hereon referred to as “NDP”). In the above sequences, “d” is used to identify the stereoisomer of the L form of the peptide. The “d” designates the dextro-rotary form of the peptide. The preferred anti-microbial and anti-inflammatory peptides may also be in the form of a dimer.

In another aspect of the invention, anti-microbial and anti-inflammatory peptide compositions are included in a topical gel or other appropriate carrier for use in prevention and treatment of wound infections.

In another aspect of the invention, anti-microbial and anti-inflammatory peptide compositions are included in a composition for locking solutions.

In another aspect of the invention, anti-microbial and anti-inflammatory peptide compositions are used in a method of treatment and prevention or infections at wound sites, and to enhance the protectiveness of locking solutions.

In another aspect of this invention, anti-microbial and anti-inflammatory peptide compositions are used to increase diuresis in peritoneal dialysis.

### BRIEF DESCRIPTION OF THE DRAWINGS

FIG. 1 illustrates a conformational structure of the KPV dimer (SEQ ID NO: 4)

FIG. 2 illustrates a Fisher projection of the KPV dimer (SEQ ID NO: 4).

FIG. 3 is a bar graph depicting the micromolar concentration of nitrite in plasma and dialysate when influenced by lipopolysaccharide (LPS) and the KPV dimer (SEQ ID NO: 4).

FIG. 4 is a bar graph depicting, in nanograms per milliliter concentration, the plasma tumor necrosis factor (TNF) under the influence of LPS and the KPV dimer (SEQ ID NO: 4).

FIG. 5 is a bar graph depicting, in nanograms per milliliter concentration the dialysate TNF under the influence of LPS and the KPV dimer (SEQ ID NO: 4).

FIG. 6 is a bar graph depicting, in milliliter per seven-hour measurement, the net ultrafiltration within the peritoneum under the influence of LPS and the KPV dimer (SEQ ID NO: 4).

FIG. 7 is a bar graph depicting, in milligrams per deciliter, the dialysate over the serum glucose under the influence of LPS and the KPV dimer (SEQ ID NO: 4).

FIG. 8 is a bar graph depicting, in milligrams per deciliter, the glucose in a dialysate under the influence of LPS and the KPV dimer (SEQ ID NO: 4).

FIG. 9 is a bar graph depicting, in picograms per milliliter concentration, the plasma tumor necrosis factor (TNF) under the influence of LPS and the KPV dimer (SEQ ID NO: 4).

FIG. 10 is a bar graph depicting, in picograms per milliliter concentration the dialysate TNF under the influence of LPS and the KPV dimer (SEQ ID NO: 4)

FIG. 11 is a bar graph depicting only minimal alteration of peritoneal transport of glucose, urea, creatine and total plasma.

FIG. 12 is a bar graph depicting the effect of α-MSH [1-1 3] (SEQ ID NO: 6), KPV and the KPV dimer (SEQ ID NO: 4) on S. aureus colony forming units (“CFU”) compared to controls. All three molecules significantly decreased S. aureus colony forming units over a broad range of peptide concentrations.

FIG. 13 is a bar graph depicting the effect of α-MSH [1-1 3] (SEQ ID NO: 6), KPV (SEQ ID NO: 1), and the KPV dimer (SEQ ID NO: 4) on S. aureus colony forming units when S. aureus' growth is enhanced by urokinase. The treatment with urokinase increases S. aureus colony formation, but that the addition of α-MSH [1-13] (SEQ ID NO: 6) or KPV (SEQ ID NO: 1) significantly inhibited this urokinase-enhancing effect.

FIG. 14 is a bar graph depicting the effect of α-MSH [1-13] (SEQ ID NO: 6), α-KPV (SEQ ID NO: 1), and the KPV dimer (SEQ ID NO: 4) C. albicans colony forming units (“CFU”) compared to controls. All three molecules significantly decreased C. albicans colony forming units over a broad range of peptide concentrations.

FIG. 15 is a bar graph depicting a comparison of antifungal activity of certain peptides and fluconazole (all 10⁻⁶M). The most effective of the peptides were those including the C-terminal amino acid sequence of KPV, for example, α-MSH [1-13] (SEQ ID NO: 6), α-MSH [6-13] (SEQ ID NO: 2), and α-MSH [11-13] (SEQ ID NO: 1).

FIG. 16(a) are photographs of plates inoculated with swabs taken from catheter sites treated with S. aureus and either the KPV dimer (SEQ ID NO: 4) or control for six days. By the sixth day, no bacterial colonies were cultured from swabs taken from animals treated with the KPV dimer (SEQ ID NO: 4).

FIG. 16(b) are photographs of plates inoculated with swabs taken from catheter sites treated with S. epidermidis and either the KPV dimer (SEQ ID NO: 4) or control for six days. By the sixth day, no bacterial colonies were cultured from swabs taken from animals treated with the KPV dimer (SEQ ID NO: 4).

FIG. 17 is a bar graph depicting the effect of KPV (SEQ ID NO: 1) on p24 release by TNF-α stimulated U1 cells. KPV (SEQ ID NO: 1) inhibited p24 release over a broad spectrum of concentrations. In this and following figures, columns or dots represent the mean and bars represent the standard deviation or confidence interval when p<0.05 (*) or p<0.01 (**).

FIG. 18 is a bar graph depicting the effect of KPV (SEQ ID NO: 1) on RT and p24 release by stimulated U1 cells. Treatment with KPV (10⁻⁵ M) inhibited HIV reverse transcriptase (RT) and p24 release from U1 cells exposed to different stimuli.

FIG. 19 is a bar graph depicting the effect of KPV (SEQ ID NO: 1) on HIV RNA in resting and PMA-stimulated U1 cells. Addition of KPV (10⁻⁵M) reduced by approximately 50% both spliced and unspliced HIV-1 RNA in PMA-stimulated U1 cells.

### DETAILED OF DESCRIPTION OF THE INVENTION

The references cited above and below are incorporated by reference as if fully set forth herein.

Preferred embodiments of the invention are directed to locking solutions, catheter care and wound site infection gels, dialysates and increasing diuresis. A locking solution is a fluid used in a catheter or other desired patent access to the body. It contains a substance that is chosen for its ability to resist clotting, blockage and other impairments of patency. A dialysate is the fluid used for fluid exchange in peritoneal dialysis. Diuresis refers to the osmotic gradient created using the invention, which limits the reabsorption of water. In this disclosure, anti-microbial is meant to include microbes including but not limited to parasites, bacterial organisms as well as viruses, fungi, and yeasts.

The invention maintains advantages over other anti-microbial therapy in that it is less likely to generate resistant microbial strains, maintains balance between natural floral strains of bacteria while helping to combat infection and it is virtually non-toxic to mammalian cells. The invention relates in particular to formulations and compositions containing the tripeptide KPV at the C-terminous and derivatives thereof. The formulations and compositions can be created forming homodimers or heterodimers of any of the peptides disclosed herein. These formulations and compositions are disclosed for use in preventing and treating infection in dialysis patients.

Gram positive organisms are by far the most common infective agents in PD, accounting for more than 70% of PD—associated peritonitis. (Okechukwu, C N, Schwartz R D Peritoneal dialysis-associated peritonitis Current Treatment Options in Infectious Disease. Biomed Central (2004) 1-22.) Coagulase-negative Staphylococcus (S. epidermidis) accounts for 30-40% of organisms isolated from PD infections (Okechukwu, C N, Schwartz R D Peritoneal dialysis-associated peritonitis Current Treatment Options in Infectious Disease. Biomed Central (2004) 1-22.) S. epidermidis is responsible for 50-70% of all medical catheter and device infections (Von Eiff, C, Proctor, R. A., Peters, G. Coagulase negative staphylococci Postgrad Med (2001) 110:63-73 whereas Staphylococcus aureus is isolated with a frequency of 14-18%. Gram negative infections in PD are much less frequent, but are often more severe in terms of hospitalization, mortality, increased rate of conversion to hemodialysis and greater risk of PD catheter loss. (Okechukwu, C N, Schwartz R D Peritoneal dialysis-associated peritonitis Current Treatment Options in Infectious Disease. Biomed Central (2004) 1-22.). The KPV dimer (SEQ ID NO: 4) is effective against Gram negative bacteria, such as E. coli and Pseudomonas aeruginosa.

In a relatively smaller number of cases of peritonitis in PD patients, either pediatric or adult, the infectious agent is a fungus. In the report of Warady (Warady B A, Baskin M, Donaldson L A, Fungal peritonitis in children receiving peritoneal dialysis: a report of the NAPRTCS Kidney Int (2000) 58:384-389) on 1592 children in which 1792 incidences of peritonitis occurred, fungal peritonitis accounted for 51 (2.9%) of the episodes. In adult PD patients, fungal peritonitis accounted for 5.1% of peritonitis episodes in a study by Bren carried out over roughly 14 years. (Bren A Fungal peritonitis in patients on continuous ambulatory peritoneal dialysis Eur J Clin Microbiol Invect Dis (1998) 17:839-843) A related study by Goldie found fungal infection in 55 (3.2%) of 1712 episodes in 704 patients over 10 years. (Goldie S J, Kiernan-Tridle L, Torres C, Gorban-Brennan N, Dunne D, Kliger A S, Finkelstein F O Fungal peritonitis in a large chronic peritoneal dialysis population: a report of 55 episodes Am J Kidney Dis (1966) 28:86-91) In these and other studies, the infectious agent was of the Candida species; primarily: 78.6% in the Warady study; and 74% in the Goldie report. Clearly, Candida sp. are important causal factors in fungal peritonitis. Although fungal infection is a less frequent cause of peritonitis in PD patients, the consequences of such infections are devastating, generally requiring catheter removal or switching to hemodialysis. (Bren A Fungal peritonitis in patients on continuous ambulatory peritoneal dialysis Eur J Clin Microbiol Invect Dis (1998) 17:839-843; Goldie S J, Kiernan-Tridle L, Torres C, Gorban-Brennan N, Dunne D, Kliger A S, Finkelstein F O Fungal peritonitis in a large chronic peritoneal dialysis population: a report of 55 episodes Am J Kidney Dis (1966) 28:86-91).

Microorganisms can proliferate at multiple sites; most commonly from contact directed or “touch contamination” in PD patients. At least one site of infection is in the peritoneal space, leading to abdominal pain, cloudy dialysis effluent and findings of pathogens on gram stains or in cultures from within the catheter. Further, the tissue tunnel or external surfaces of the catheter are common infection points from touch contamination. Infection at the tissue tunnel is most commonly due to migration from skin sites. From this information, there are at least three critical sites of potential infection prevention and control, sites that can be accessed directly by the patient or caregiver. These sites include the peritoneal dialysis fluid within the peritoneal catheter and at the catheter exit site.

Infection control applied to these sites can be used to prevent and/or to treat common infections in PD. Control of such infection can, therefore, be approached by including anti-infective agents in PD fluid, in locking solutions, and in medications used to treat catheter exit sites.

Treatment of these infections and prevention of infection at these vulnerable sites is disclosed herein via anti-microbial and anti-inflammatory peptides. The preferred antimicrobial and anti-inflammatory peptides are the C-terminal KPV moiety of α-MSH (SEQ ID NO: 1), the KPV dimer (SEQ ID NO: 4) and Phe7 (SEQ ID NO: 5).

KPV (SEQ ID NO: 1) is commonly associated with α-MSH (SEQ ID NO: 6) as KPV (SEQ ID NO: 1) forms the C-terminus tripeptide of α-MSH (SEQ ID NO: 6). Many studies have been performed on KPV (SEQ ID NO: 1) and α-MSH (SEQ ID NO: 6), the results of which are useful to an understanding of the disclosure herein.

Unmodified α-MSH (SEQ ID NO: 6) is an ancient, thirteen amino-acid peptide produced by post-translational processing of the larger precursor molecule propiomelanocortin. It shares the same 1-13 amino acid sequence with adrenocorticotropic hormone (“ACTH”) (SEQ ID NO: 8), also derived from propiomelanocortin. α-MSH (SEQ ID NO: 6) is secreted by many cell types, including pituitary cells, monocytes, melanocytes, and keratinocytes. It can be found in the skin of rats, in the human epidermis, or in the mucosal barrier of the gastrointestinal tract in intact and hypophysectomized rats. (See e.g., Eberie, A. N., “The Melanotrophins,” Karger, Basel, Switzerland (1998); Lipton, J. M., et. al., “Anti-inflammatory Influence of the Neuroimmunomodulator α-MSH,” Immunol. Today 18, 140-145 (1997); Thody, A. J., et. al., “MSH Peptides are Present in Mammalian Skin,” Peptides 4, 813-815 (1983); Fox, J. A., et. al., “Immunoreactive α-Melanocyte Stimulating Hormone, Its Distribution in the Gastrointestinal Tract of Intact and Hypophysectomized Rats,” Life. Sci. 18, 2127-2132 (1981).)

Alpha-MSH (SEQ ID NO: 6) is known to have potent antipyretic, anti-microbial and anti-inflammatory properties, yet extremely low toxicity, and α-MSH can reduce production of the host cells' pro-inflammatory mediators in vitro, and can reduce production of local and systemic reactions in animal models for inflammation. The “core” α-MSH sequence, Met-Glu-His-Phe-Arg-Trp-Gly (SEQ. ID NO. 9) for example, has learning and memory behavioral effects but little antipyretic and anti-inflammatory activity. In contrast, the active message sequence for the antipyretic and anti-inflammatory activities resides in the carboxy-terminal KPV (SEQ ID NO: 1) sequence of α-MSH (SEQ ID NO: 6). This tripeptide has activities in vitro and in vivo that parallel but are more potent than those of the parent molecule. The anti-inflammatory activity of α-MSH (SEQ ID NO: 6) and KPV (SEQ ID NO: 1) are disclosed in the following two patents and numerous references, which are hereby incorporated by reference: U.S. Pat. No. 5,028,592, issued on Jul. 2, 1991 to Lipton, J. M., entitled “ANTIPYRETIC AND ANTI-INFLAMMATORY LYS PRO VAL COMPOSITIONS AND METHOD OF USE;” U.S. Pat. No. 5,157,023, issued on Oct. 20, 1992 to Lipton, J. M., entitled “ANTIPYRETIC AND ANTI-INFLAMMATORY LYS PRO VAL COMPOSITIONS AND METHOD OF USE;” see also Catania, A., et. al. Melanocyte Stimulating Hormone in the Modulation of Host Reactions,” Endocr. Rev. 14, 564-576 (1993); Lipton, J. M., et al., “Anti-inflammatory Influence of the Neuroimmunomodulator of α-MSH, Immunol.” Today 18, 140-145 (1997); Rajora, N., et. al., “α-MSH Production Receptors and Influence on Neopterin, in a Human Monocyte/macrophage site e Cell” Line, J. Leukoc. Biol. 59, 248-253 (1996); Star, R. A., et. al., “Evidence of Autocrine Modulation of Macrophage Nitric Oxide Synthase by α-MSH,” Proc. Nat'l. Acad. Sci. (USA) 92, 8015-8020 (1995); Lipton, J. M., et. al., “Anti-inflammatory Effects of the Neuropeptide α-MSH in Acute Chronic and Systemic inflammation,” Ann. N.Y. Acad. Sci. 741, 137-148 (1994); Fajora, N., et. al., “α-MSH Modulates Local and Circulating Tumor Necrosis Factor α in Experimental Brain Inflammation α-MSH Modulates Local and Circulating Tumor Necrosis Factor-α in Experimental Brain Inflammation,” J. Neurosci, 17, 2181-2186 (1995); Richards, D. B., et. al., “Effect of α-MSH (11-13) (lysine-proline-valine) on Fever in the Rabbit,” Peptides 5, 815-817 (1984); Hiltz, M. E., et. al., “Anti-inflammatory Activity of a COOH-terminal Fragment of the Neuropeptide α-MSH,” FASEB J. 3, 2282-2284 (1989).

A preferred embodiment of the invention is used to treat and prevent infection and inflammation using a peptide selected from the group of peptides with an amino acid sequence consisting of KPV (SEQ. ID NO.1), HFRWGKPV (SEQ. ID NO. 2), HdNIeRWGKPV (SEQ. ID NO. 3), a KPV dimer VPKC-s-s-CKPV (SEQ ID NO: 4), SYSMEHdFRWGKPV (SEQ ID NO: 5), SYSMEHFRWGKPV (SEQ ID NO: 6), and [Nle₄ dPhe₇]α-MSH (SEQ ID NO: 7).

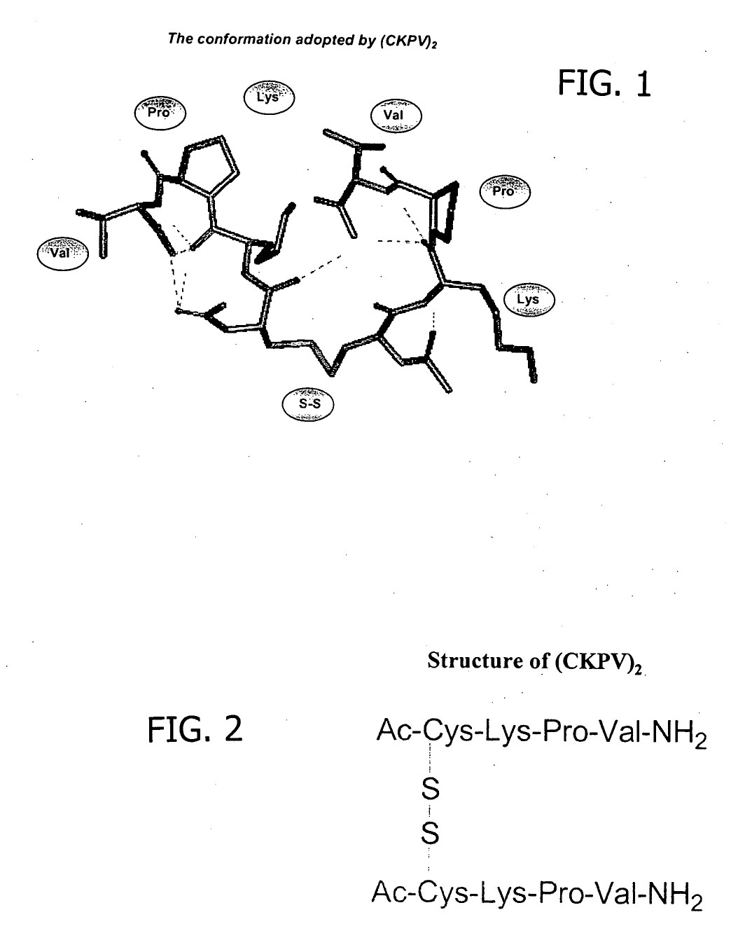

The KPV dimer (SEQ ID NO: 4) is illustrated in FIGS. 1 and 2 where FIG. 1 illustrates a conformational structure and FIG. 2 is a Fisher projection of the same molecule. The KPV dimer (SEQ ID NO: 4) is formed when a linker links the N-terminals of two KPV (SEQ ID NO: 4) peptides. For example, VPKC-s-s-CKPV (SEQ ID NO: 4), one kind of dimer, is formed by adding a cysteine at the N-terminal of KPV peptide and allowing the cysteines of two CKPV peptides to form a disulfide bond (-s-s-). In other words, VPKC-s-s-CKPV is formed when a-Cys-s-s-Cys- linker joins the two KPV (SEQ ID NO: 1) peptides. The linker can be modified to any kind of chemical bond that links the N-terminals of two KPV (SEQ ID NO: 1) peptides together. The different variations of linkers create a modified KPV dimer (SEQ ID NO: 4). Preferred modified KPV dimer (SEQ ID NO: 4) linkages may be selected from the group consisting of -Cys-s-s-Cys-, -dCys-s-s-Cys-, -Pen- s-s-Cys-, -Pen- s-s-dCys-, -dPen-s-s-Cys-, -dPen-s-s-dCys-, -dPen-s-s-DPen-, -Pen-s-s-Pen-, -hCys-s-s-Cys-, -hCys-s-s-dCys-, -hCys-s-s-hCys-, -dhCys-s-s-dhCys-, -dhCys- s-s-hCys-, -hCys-s-s-Pen-, -hCys-s-s-dPen-, or -dhCys-s-s-dPen-. It is more preferred that the linker be -Cys-Cys-. The term “Pen” refers to Penicillamine. The Term “Cys” refers to Cysteine. The Term “hcys” refers to Homocysteine. The prefix “d” refers to the dextro-form of an amino acid. Accordingly, it is preferred that the KPV dimer (SEQ ID NO: 4) be VPK-Cys-ss-Cys-KPV (SEQ ID NO: 4), VPK-dCys-s-s-Cys-KPV (SEQ ID NO: 10)-s-s-(SEQ ID NO: 4), VPK-Pen-s-s-Cys-KPV (SEQ ID NO: 4), VPK-dPen-s-s-dCys-KPV (SEQ ID NO: 10), VPK-dPen-s-s-Cys-KPV (SEQ ID NO: 4), VPK-dPen-s-s-dCys-KPV (SEQ ID NO: 10), VPK-dPen-s-s-dPen-KPV, VPK-Pen-s-s-Pen-KPV VPK-hCys-s-s-Cys-KPV (SEQ ID NO: 11)-s-s-(SEQ ID NO: 4), VPK-hCys-s-s-dCys-KPV (SEQ ID NO: 11)-s-s-(SEQ ID NO: 10), VPK-hCys-s-s-hCys-KPV (SEQ ID NO: 11), VPK-DhCys-s-s-DhCys-KPV (SEQ ID NO: 12), VPK-dhCys-s-s-hCys-KPV (SEQ ID NO: 12)-s-s-(SEQ ID NO: 11), VPK-hCys-s-s-Pen-KPV (SEQ ID NO: 11), VPK-hCys-ss-dPen-KPV (SEQ ID NO: 11), or VPK-dhCys-s-s-DPen-KPV (SEQ ID NO: 12).

Homocysteine is disclosed here as an example of a possible linker. However, recent studies have determined that homocysteine may be a risk factor for many disease conditions, including heart disease, stroke, and cleft palate. (http://www.homocysteine.net/pages/homocysteine/1/abouthcy.html.) Consequently, the risk of homocysteine use should be considered in any specific indication.

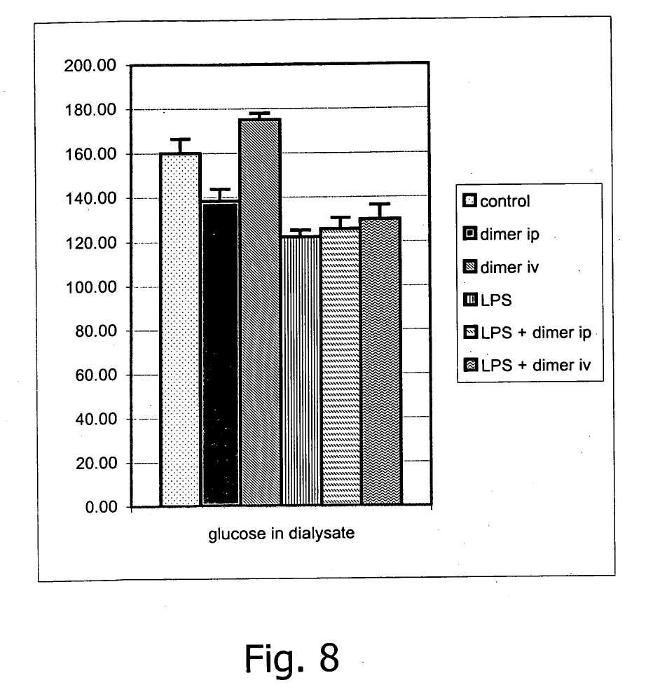

Previous research has shown that the immunomodulatory peptide α-MSH composed of thirteen amino acids, had remarkable antimicrobial influences (Cutuli, M. D.; Cristiani, S.; Lipton, J. M.; Catania, A. Antimicrobial effects of α-MSH peptides. J Leukoc Biol. (2000) 67:233-239; Catania, A.; Cutuli, M. G.; Garofalo, L.; Carlin, A.; Airaghi, L.; Barcellini, W.; Lipton, J. M. The Neuropeptide α-MSH in the Host Defense. Ann N Y Acad Sci (2000) 917:227-231). Evidence indicates that the natural α-MSH (1-13) (SEQ ID NO: 6) peptide and its carboxy-terminal tripeptide α-MSH (11-13) (SEQ ID NO: 1) effectively reduce viability of Candida albicans and Staphylococcus aureus. Because the central tetrapeptide His-Phe-Arg-Trp (SEQ ID NO: 13) was not required for the candidacidal effect of α-MSH (SEQ ID NO: 6), focus of the inventors was placed on the C-terminal tripeptide Lys-Pro-Val (SEQ ID NO: 1) which exerts anti-inflammatory influences similar to those of the parent molecule (Hiltz, M. E.; Lipton, J. M. Anti-inflammatory activity of a COOH-terminal fragment of the neuropeptide α-MSH. FASEB J (1980) 3:2282-2284; Lipton, J. M.; Catania, A. Anti-inflammatory actions of the neuroimmunomodulator α-MSH. Immunol Today (1997) 18:140-145) and, in related experiments, showed substantial candidacidal influences. The dimer of this tripeptide (FIG. 8) showed excellent candidacidal effects and, therefore, investigations based on this molecule were expanded.

The KPV dimer (SEQ ID NO: 4) was tested for potential activity against Candida sp., including strains of C. krusei and C. glabrata. Such strains are prevalent in an immunocompromised host and represent an emerging therapeutic problem in that they are generally resistant to the currently available antimycotic agents. The combined results indicate that the KPV dimer (SEQ ID NO: 4) has important antimycotic activities and these results, along with the results of recent clinical trials indicating remarkable safety of and tolerability to this molecule, support the use of this agent in control of infection in peritoneal dialysis. Moreover, similar successful results of positive activity have been obtained using the KPV dimer (SEQ ID NO: 4) in HIV infected individuals. This activity, which is more functionally disclosed in the examples, clearly shows a cross kingdom efficaciousness of the KPV dimer (SEQ ID NO: 4) in treating infection.

A functional equivalent is defined as amino acid sequences with a C-terminus KPV that are functionally equivalent in terms of anti-inflammatory and anti-microbial activity KPV (SEQ ID NO: 1), such as, but not limited to, VPKC-s-s-CKPV, HFRWGKPV (SEQ. ID NO. 2) and SYSMEHRdFWGKPV (SEQ ID NO: 5) HdNleRWGKPV (SEQ ID NO: 3). Although the specific amino acid sequences described here are effective, it is clear to those familiar with the art that amino acids can be substituted in the amino acid sequence or deleted without altering the effectiveness of the peptides. For example, modifications of the KPV dimer (SEQ ID NO: 4), some of which are described above, are functionally equivalent. For example, a stable analog of α-MSH, [Nle4 dPhe7]-α-MSH, hereon “NDP,” (SEQ ID NO: 7) which is known to have marked biological activity on melanocytes and melanoma cells, is approximately ten times more potent than the parent peptide in reducing fever. (Holdeman, M. and Lipton, J. M., Antipyretic Activity of a Potent α-MSH Analog, Peptides 6, 273-5 (1985). Further, adding amino acids to the C-terminal α-MSH (11-13) (SEQ ID NO: 1) sequence can reduce or enhance antipyretic potency (Deeter, L. B.; Martin, L. W.; Lipton, J. M., Antipyretic Properties of Centrally Administered α-MSH Fragments in the Rabbit, Peptides 9, 1285-8 (1989). Addition of glycine to form the 10-13 sequence slightly decreased potency; the 9-13 sequence was almost devoid of activity, whereas the potency of the 8-13 sequence was greater than that of the 11-13 sequence. It is known that [dK11] α-MSH (11-13) has the same general potency as the L-form of the tripeptide α-MSH 11-13 (SEQ ID NO: 1). (Hiltz, M. E.; Catania, A.; Lipton, J. M., Anti-inflammatory Activity of α-MSH (11-13) Analogs: Influences of Alterations in Stereochemistry, Peptides 12, 767-71 (1991).) Substitution with the D-form of valine in position 13 or with the D-form of lysine at position 11 plus the D-form of valine at position 13 resulted in greater anti-inflammatory activity than with the L-form tripeptide. (Id.) These examples indicate that alterations in the amino acid characteristics of the peptides can influence activity of the peptides or have little effect, depending upon the nature of the manipulation.

It is further understood that each of the sequences disclosed herein are capable of forming either homodimers or heterodimers. For example, Phe7 may be used in a homodimer formation in which the dimer would be VPKGWRdFHEMSYSC-s-s-CSYSMEHdFRWGKPV (SEQ ID NO: 16)

It is also understood that functional equivalents may be obtained by substitution of amino acids having similar hydropathic values. Thus, for example, isoleucine and leucine, which have a hydropathic index +4.5 and +3.8, respectively, can be substituted for valine, which has a hydropathic index of +4.2, and still obtain a protein having like biological activity. Alternatively, at the other end of the scale, lysine (−3.9) can be substituted for arginine (−4.5), and so on. In general, it is believed that amino acids can be successfully substituted where such amino acid has a hydropathic score of within about ±1 hydropathic index unit of the replaced amino acid.

The anti-microbial properties and functional equivalents can be measured through their inhibitory effect on the colony forming units in bacteria or fungi, or through their inhibitory effect on virus expression or transcription, as disclosed in the examples herein. Moreover, the multiple organism cidal activity and cross kingdom cidal activity of the peptides disclosed herein are important to the dialysis patient.

Infections are not confined to a single cause. Multiple organism infections are common in dialysis patients. For treatment of these infections, anti-microbial and anti-inflammatory peptides may be applied locally to the site of the infection and/or inflammation by methods known in the art. For example, modified anti-microbial and anti-inflammatory peptides may be dissolved in solutions such as phosphate buffer saline, hyalurinate, methylcellulose, carboxymethicellulose, or ethanol. Solvated anti-microbial and anti-inflammatory peptides may then be combined with vehicles such as solutions that are biocompatible. Topical ointments, creams, gels, aerosol sprays, suppositories, liquid solutions and absorbent materials are contemplated.

In one preferred embodiment of the invention, dialysate is prepared using anti-microbial and anti-inflammatory peptides containing KPV at the C-terminus. The anti-microbial and anti-inflammatory peptides are therapeutically effective within the range of nanomolar to millimolar concentrations in the dialysate. In different embodiments of the invention, the dialysates contain glucose at least about 1 to 5 percent; 2 to 3 percent; and at 2.75 percent; sodium chloride at least about 3 to 6 grams; 4-5 grams; and at 4.5 grams; calcium chloride at least about 0.01 to 0.3 grams; 0.05 to 0.2; and at 0.1 grams; magnesium chloride at 0.005 to 0.12 grams; 0.008 to 0.05; and at 0.0625 grams; glucose monohydrate at least about 25 to 50 grams; 35 to 40 grams; and at 37.5 grams; and anti microbial and anti-inflammatory peptides in at least about the millimolar to nanomolar range such as 1×10-3 to 1×10⁻⁹M and at least about 2×10⁻³ to 2×10⁻¹¹M and concentrations in-between. It is important to note that glucose concentration varies with an individual patient's needs. It is supplied in 1.5 to 4.25% and in bags of 1.5.2, 2.5 or 3 liters (pediatric) and up to 10 liters for adults. The peptide is preferably supplied in lyophilized sterile vials and is added to the fluid through standard aseptic techniques such as re-suspension of the lyophilized peptide and with sterile water, withdrawing the mixture into an appropriate size syringe and injecting the mixture into the fluid. For purposes of preservation, the peptide may be kept dry until the time of use. It is also contemplated that the dialysate may contain the anti-microbial and anti-inflammatory peptides in solution ready for peritoneal dialysis.

To meet the ultrafiltration requirements of patients on peritoneal dialysis, the peritoneal dialysate is typically administered hyperosmolar relative to plasma to create an osmotic gradient that favors net movement of water into the peritoneal cavity; this is the nature of the ability of peritoneal dialysis to accomplish cleansing. In commercially available peritoneal dialysates, the sugar glucose serves as the osmotic agent that enhances ultrafiltration. Available concentrations range from 1.5% to 4.25% dextrose or glucose. Over time, the osmolality of the dialysate declines as a result of water moving into the peritoneal cavity and of absorption of dialysate sugars. The absorption of glucose contributes substantially to the calorie intake of patients on continuous peritoneal dialysis.

Over time, this carbohydrate load is thought to contribute to progressive obesity, hypertriglyceridemia and decreased nutrition as a result of loss of appetite and decreased protein intake. It is believed that the addition of anti-microbial and anti-inflammatory peptides in the dialysate adds to the hyperosmolarity of the dialysate so as to necessitate less glucose. In addition, the high glucose concentrations and high osmolality of currently available solutions has negative effects on the function of leukocytes, peritoneal macrophages and mesothelial cells.

Electrolytes also play an important role in dialysis. For example, the sodium concentration in the ultrafiltrate during peritoneal dialysis is usually less than that of extracellular fluid, so there is a tendency toward water loss and development of hypernatremia. Commercially available peritoneal dialysates have a sodium concentration of 132 mEq/L to compensate for this tendency toward dehydration. The effect is more pronounced with increasing frequency of exchanges and with increasing dialysate glucose concentrations. Use of the more hypertonic solutions and frequent cycling can result in significant dehydration and hypernatremia, that leads to thirst. As a result of stimulated thirst, water intake and weight may increase, resulting in a vicious cycle. Potassium is cleared by peritoneal dialysis at a rate similar to that of urea. With CAPD and 10 L of drainage per day, approximately 35 to 46 mEq of potassium is removed per day. Daily potassium intake is usually greater than this, yet significant hyperkalemia is uncommon in these patients. Presumably, potassium balance is maintained by increased colonic secretion of potassium and by some residual renal excretion or Residual Renal Function (RRF). Given these considerations, potassium is not routinely added to the dialysate.

The buffer present in most commercially available peritoneal dialysate solutions is lactate. In patients with normal hepatic function, lactate is rapidly converted to bicarbonate, so that each mM of lactate absorbed generates one mM of bicarbonate. Even with the most aggressive peritoneal dialysis there is no appreciable accumulation of circulating lactate. The rapid metabolism of lactate to bicarbonate maintains the high dialysate-plasma lactate gradient necessary for continued absorption. The pH of commercially available peritoneal dialysis solutions is purposely made acidic by adding hydrochloric acid to prevent hydrocarbons, glucose or dextrose for example, from caramelizing during the sterilization procedure. Once instilled, the pH of the solution rises to values greater than 7.0.

There is some evidence that the acidic pH of the dialysate, in addition to the high osmolality, may impair the host's peritoneal defenses. For this reason, addition of anti-microbial and anti-inflammatory peptides avoid complications due to compromising host defenses by decreasing or preventing inflammation to the peritoneal membrane.

To avoid negative calcium balance-and possibly to suppress circulating parathyroid hormone-commercially available peritoneal dialysis solutions evolved to have a calcium concentration that is equal to or slightly greater than the ionized concentration in the serum of most patients. As a result, there is a net calcium absorption in most patients treated with a conventional chronic ambulatory peritoneal dialysis regimen. As the use of calcium-containing phosphate binders has increased, hypercalcemia has become a common problem when utilizing the 3.5 mEq/L calcium dialysate. This complication has been particularly common in patients treated with peritoneal dialysis, since they have a much greater incidence of adynamic bone disease than do hemodialysis patients. In fact, the continual positive calcium balance associated with the 3.5-mEq/L solution has been suggested to be a contributing factor in the development of adynamic bone disease.

The low bone turnover state typical of this disorder impairs accrual of administered calcium, contributing to the development of hypercalcemia. As a result, there has been increased interest in using a strategy similar to that employed in hemodialysis, namely, lowering the calcium content of the dialysate. A preferred embodiment of this invention will similarly lower the calcium content of the dialysate.

In one embodiment of the invention, the invention may be used as an adjunct to commercially available dialysates wherein the anti-microbial and anti-inflammatory peptides may be added to the dialysate. It is further contemplated that a dialysate containing anti-microbial and anti-inflammatory peptides may be developed to avoid the addition of anti-microbial and anti-inflammatory peptides to an already developed dialysate.

Locking solutions are also commercially available “Heparin locks” are the most common locking solution in catheters in general and heparin is a common component of locking solutions in dialysis. In another embodiment of the invention, a locking solution containing anti-microbial and anti-inflammatory peptides is used for both peritoneal dialysis and hemodialysis. Parenteral anti-coagulants such as heparin, danaparoid and lepirudin are contemplated for use in the invention. A locking solution may contain anti-microbial and anti-inflammatory peptides preferably in an amount in the range of millimolar to nanomolar amounts.

For catheter care, the anti-microbial and anti-inflammatory peptides will be mixed in a composition with a non-toxic, biologically compatible carrier prior to administration. Usually, this will be an aqueous solution, such as normal saline or phosphate-buffered saline (PBS), Ringer's solution, Ringer's lactate or any isotonic physiologically acceptable solution for administration by the chosen means. Preferably, the solution is manufactured and packaged under current Good Manufacturing Processes (GMP's) as approved by the FDA.

In another embodiment of the invention, anti-microbial and anti-inflammatory peptides may be supplied in a kit for use in dialysis. The anti-microbial and anti-inflammatory peptides may be presented in a lyophilized form in a sterile vial or other container composed of metal plastic or glass and including envelope type containers. The peptide may be to nanomolar range of at least about 1×10⁻³ to 1×10⁻⁹M and at least about 2×10⁻³ to 2×10⁻¹¹M1 and concentrations in-between. The kit may also contain a sterile solvent in a similar type of container used for the peptide, but adjusted for volume, for the anti-microbial and anti-inflammatory peptides to be dissolved in. The solvent may be selected depending on the type of kit used. For example, a kit designed for use with a catheter may contain a solution containing 1200 to 5000 units of heparin in sterile saline or sterile water. Heparin concentrations and heparinization are well known in the art. On the other hand, a kit for use with a dialysate may contain a solvent for the anti-microbial and anti-inflammatory peptides that is without heparin. Solvents such as Ringer's lactate, D5W, saline, sterile water, de-ionized water and the like, are appropriate. The kit will further comprise an applicator selected from the group consisting of spatulas, cotton-tip applicators, syringes, spray bottles, moist towels, droppers. The kit may also include instructions for use.

Each kit may contain any of a number of devices for transferring the dissolved anti-microbial and anti-inflammatory peptides into an end receptacle. A transferring device is understood to mean a syringe, pump, pipette or any device used for aseptic transfer of a substance. The end receptacle is understood to mean the receptacle for the dialysate or a catheter used in dialysis. The end receptacle could also mean a port or other access point for introduction of medicines on a dialysis machine in hemodialysis. The dissolved anti-microbial and anti-inflammatory peptides may be added directly to the dialysate or transferred into the catheter in peritoneal dialysis and may be used in hemodialysis during a hemodialysis treatment by infiltrating the dissolved anti-microbial and anti-inflammatory peptides during a hemodialysis exchange.

Although IV and IP administration are preferred, in another embodiment of the above invention, modified anti-microbial and anti-inflammatory peptides may be administered orally. Each oral composition according to the present invention may additionally comprise inert constituents including biologically compatible carriers, dilutents, fillers, wetting agents, suspending agents, solubilizing or emulsifying agents, salts, flavoring agents, sweeteners, aroma ingredients or combinations thereof, as is well-known in the art. Liquid dosage forms may include a liposome solution containing the liquid dosage form. As known by those ordinarily skilled in the art, suitable forms for suspending liposomes include emulsions, pastes, granules, compact or instantized powders, suspensions, solutions, syrups, and elixirs containing inert dilutents, such as purified water.

Tablets or capsules may be formulated in accordance with conventional procedures employing biologically compatible solid carriers well known in the art. For example, a pharmaceutical preparation may contain the composition dissolved in the form of a starch capsule, or hard or soft gelatin capsule which is coated with one or several polymer films, in accordance with U.S. Pat. No. 6,204,243 which is fully incorporated as if fully set out herein. The choice and usage of appropriate polymers, including additional materials such as softeners and pore-forming agents, control the site of dissolution of the capsule and the release of solution containing the active agent.

Preparation of the composition may also include dissolving the composition in a solvent, which is suitable for encapsulation into starch or gelatin capsules, or in a mixture of several solvents and, optionally, solubilizers and/or other excipients. The solution is then filled into starch capsules, or hard or soft gelatin capsules in a measured dose, the capsules are sealed, and the capsules are coated with a solution or dispersion of a polymer or polymer mixture and dried. The coating procedure may be repeated once or several times.

The solvents that are appropriate for dissolving the active agent are those that are biologically compatible with the host subject and in which the composition dissolves. Examples of these are ethanol, 1,2-propylene glycol, glycerol, polyethylene glycol 300/400, benzyl alcohol, medium-chained triglycerides and vegetable oils.

Medicament excipients may be added to the solution. Examples of such excipients are mono and/or di-fatty acid glycerides, sorbitan fatty acid esters, polysorbates, lecithin, sodium lauryl sulphate, sodium dioctylsulphosuccinate, aerosol and water-soluble cellulose derivatives. Mixtures of solvents and excipients may also be used. See, in general, Remingtons's Pharmaceutical Sciences (18th Ed. Mack Publishing Co. 1990) or Remington's: The Science and Practice of Pharmacy by Alphonso R. Gennero (Lippincott Williams & Wilkins 2003).

When administration is parenteral, injectable pharmaceuticals may be prepared in conventional forms, as aqueous or non-aqueous solutions or suspensions; as solid forms suitable for solution or suspension in liquid prior to injection; or as emulsions. Examples of non-aqueous solvents are propylene glycol, polyethylene glycol, vegetable oils such as olive oil, and injectable organic esters such as ethyl oleate. Other suitable excipients are water, saline, dextrose, mannitol, lactose, lecithin, albumin, sodium glutamate, cysteine hydrochloride, or the like. In addition, the injectable pharmaceutical compositions may contain minor amounts of non-toxic auxiliary substances, such as wetting agents, pH buffering agents, and the like. If desired, absorption-enhancing preparations (e.g., liposomes) may be used.

Topical administration for wound infections include a composition to be placed at the wound site and within tunnel infections. In this embodiment of the invention, the anti-microbial and anti-inflammatory peptides are at least about 0.01 to 0.9% in a standard topical gel. Gel formulations are well known in the art. (See, in general, Remingtons's Pharmaceutical Sciences (18th Ed. Mack Publishing Co. 1990) or Remington's: The Science and Practice of Pharmacy by Alphonso R. Gennero (Lippincott Williams & Wilkins 2003).) The gel can also be supplied in 0.025%, 0.05%, 0.075% and 0.09 % by weight in a standard topical gel. In one preferred embodiment of a gel formulation, the gel comprises: carbopol 974P, propylparaben, methylparaben, propylene glycol, EDTA, 2M NaOH solution, and sterile water for injection. Dressing changes are at the discretion of the wound care specialist and are contemplated to be effective at each dialysate exchange for peritoneal dialysis and three to four times a day for other applications.

Peptides of this invention are preferably —N-acetylated and C-amidated. The peptides may be prepared by solid-phase peptide synthesis and purified by reversed-phase high performance liquid chromatography. The KPV dimer (SEQ ID NO: 4) can be chemically represented as Val-Pro-Lys-AcCys-s-s-CysAc-Lys-Pro-Val or VPKC-s-s-CKPV and in this disclosure is identified as SEQ ID NO: 4. The KPV dimer (SEQ ID NO: 4) is formed by adding cysteines at the N-terminal of KPV (SEQ ID NO: 1) peptide and allowing the cysteines of two CKPV peptides to form a disulfide bond. As shown in FIG. 1 the molecular conformation of the KPV dimer (SEQ ID NO: 4) was determined through molecular modeling techniques. The molecular modeling studies were performed using the SYBYL software version 6.2 running on a Silicon Graphic Indingo 2 workstation. FIG. 1 represents a conformational structure of the KPV dimer (SEQ ID NO: 4). The conformational study showed that the KPV dimer (SEQ ID NO: 4) adopts a structure well organized and stabilized by intra-molecular hydrogen bounds. The tertiary structure of the dimer is folded and amino acids are well protected. It also resembles a cyclic peptide with a beta-turn.

### EXAMPLE I

### Dialysate Containing Anti-Microbial and Anti-Inflammatory Peptides Used in Mice

At time 0, 15 ml of dialysis fluid was administered to mice with or without lipopolysaccharide (LPS), a known irritant from bacterial cell walls, and with or without intravenous (IV) KPV dimer (SEQ ID NO: 4) or NDP (SEQ ID NO: 7) administration. The dosage of all peptides administered in the fluid, (KPV dimer (SEQ ID NO: 4) or NDP (SEQ ID NO: 7)), was 10⁻⁵ M. The same amount was given IP. Seven hours later the animals were sacrificed and blood and peritoneal dialysis fluid removed for analysis.

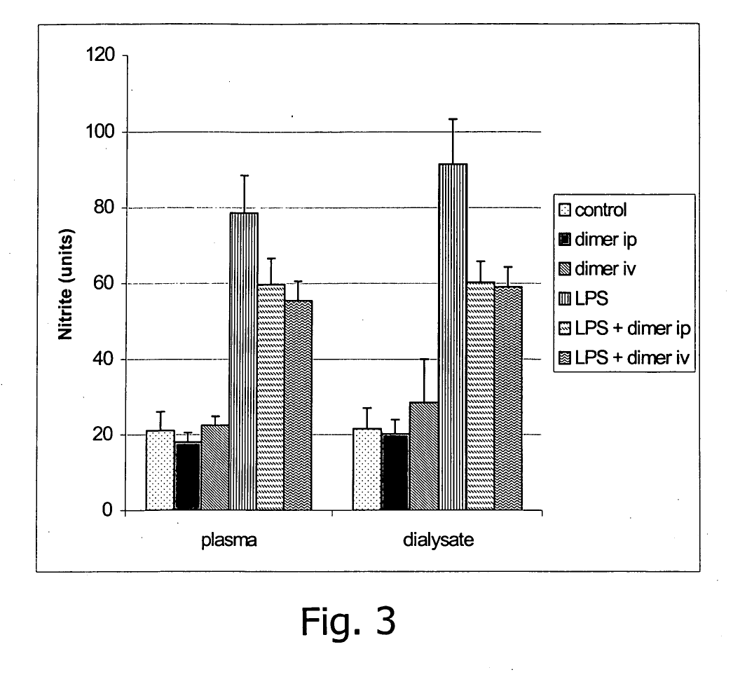

Important findings relate to nitrite and are shown in FIG. 3. Nitrite is used as an estimate of nitric oxide production in plasma and dialysate. This is a method to estimate inflammation in the plasma compartment and the peritoneal sack. These compartments are intimately related and changes in concentration generally occur in parallel. The graph depicted in FIG. 3 further identifies sub-units of plasma and dialysate. The units on the left axis are micromolar. The KPV dimer (SEQ ID NO: 4) given IV or into the dialysate had no effect on normal nitric oxide production in either compartment. LPS in the dialysate resulted in marked increases in nitric oxide production in both compartments. This increase in inflammatory response was modulated by the dimer, whether it was given intravenously or included in the dialysate.

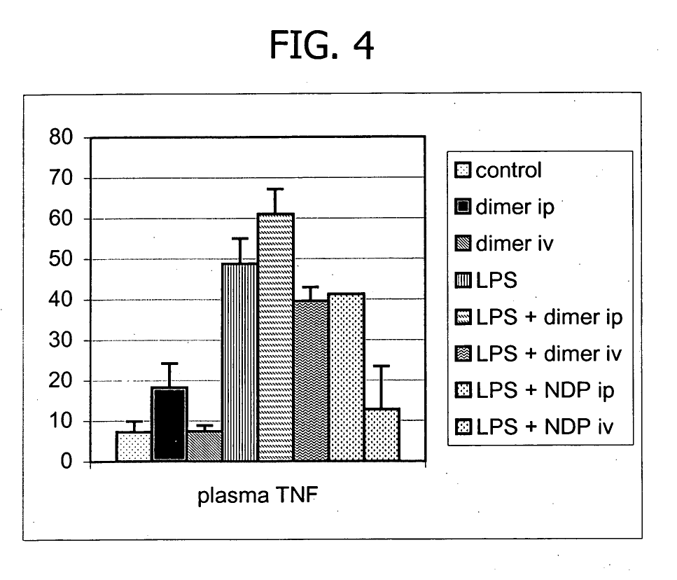

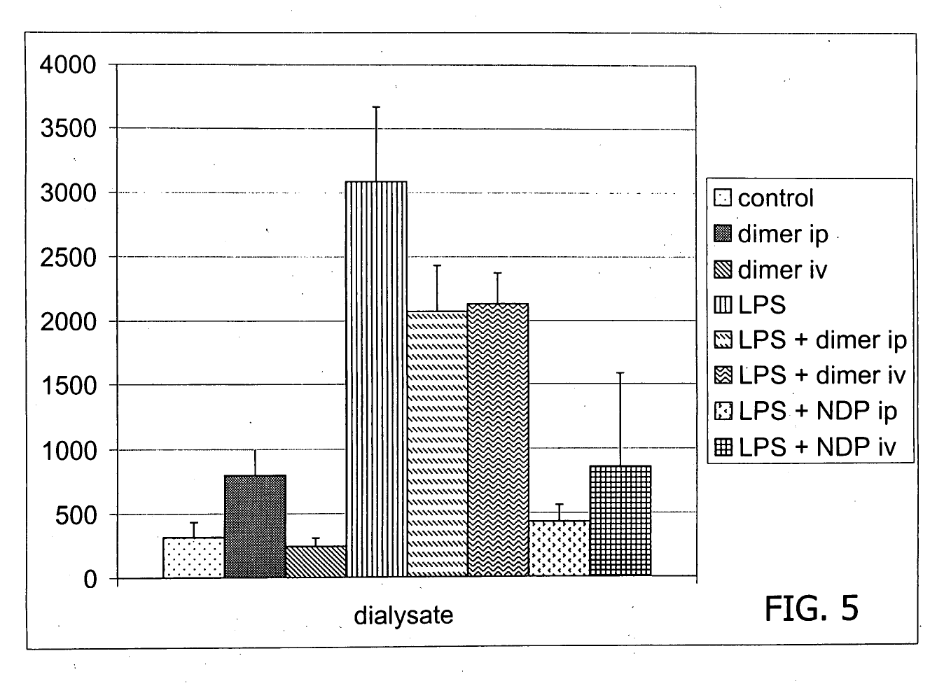

The experiment also revealed important data related to Tumor Necrosis Factor-α (“TNF”). This data is illustrated in FIGS. 4 and 5. FIG. 4 relates to plasma TNF (left axis is nanogram/ml) and FIG. 5 relates to dialysate TNF (left axis form 0-4000 ng/ml). Again, NDP refers to the alpha MSH molecule with substitution of norleucine in the fourth position and dPhe in the seventh position.

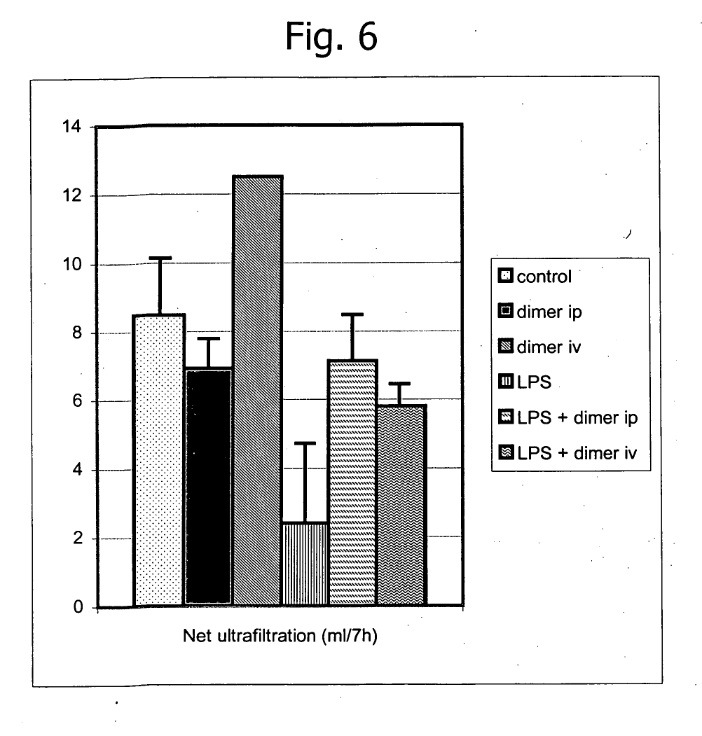

The net ultrafiltration (ml/7hr) is depicted in FIG. 6. In this case the volume of fluid retrieved at the end of the 7 hr period was subtracted from the initial 15 ml volume. The dimer given IV caused a loss of about 12 ml of fluid from the cavity. LPS caused marked retention of fluid. The dimer given by IP or IV injection increased the loss of fluid from the cavity, substantially reducing the water retaining effect of the LPS. This result led to the unexpected realization that use of the invention increased diuresis over the dialysate without the peptides.

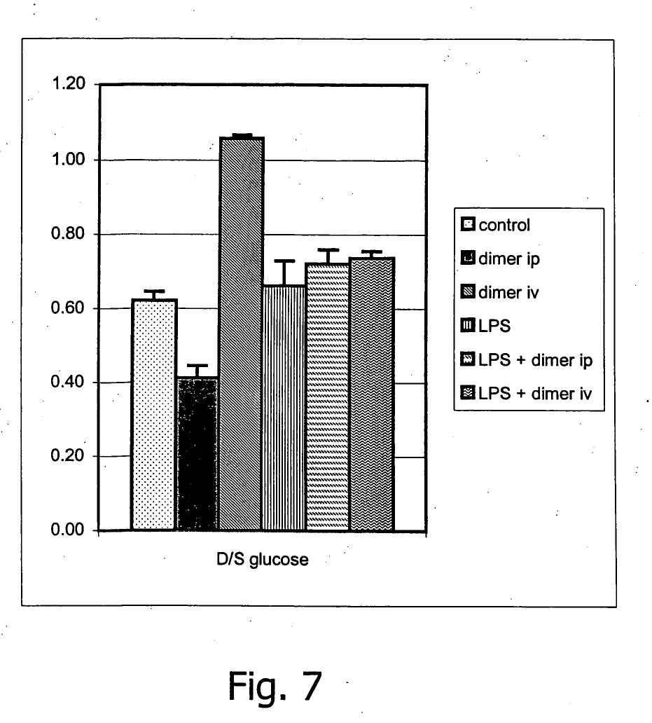

The figure on D/S glucose (dialysate glucose over serum glucose), FIG. 7, reflects the same effect of the dimer on diuresis. This is an unexpected and positive feature for patients as use of the invention results in an increase in diuresis as measured by the ratio of dialysate glucose over serum glucose. The logic is similar when measuring the glucose in dialysate illustrated in FIG. 8. With the reduction of water, the concentration of the glucose is increased. LPS is known to have the opposite effect.

### EXAMPLE II

### In vivo Dialysis Fluid Study

To demonstrate the protective effect of the KPV dimer on peritoneal and systemic inflammation caused by endotoxin in rats during PD, an in vivo dialysis study was performed. Similar to the above Example, the goal of the study was to learn if the KPV dimer might be useful in controlling inflammation associated with endotoxin in the peritoneal space when the peptide is incorporated in dialysis fluid.

Acute peritonitis was induced in male Wistar rats (200-220 g) by adding lipolysaccharide (LPS, 5 μg/ml) to the dialysis fluid. the KPV dimer was either added to PD fluid or given iv. Rats (n=6 per group) received either: a) 15 ml dialysis fluid; or b) dialysis fluid plus the KPV dimer 140 μg; or c) dialysis fluid plus LPS; or c) dialysis fluid plus LPS 5 μg/ml and the KPV dimer 140 μg; or d) dialysis fluid plus LPS 5 μg/ml and the KPV dimer 140 μg given iv. After 7 hours rats were sacrificed under ether anesthesia and the abdomen incised.

Peritoneal fluid was removed and examined for nitrite and TNFα concentrations in picograms per milliliter. The effect of the KPV dimer on peritoneal transport of small and large molecules was evaluated by measuring dialysate/serum ratio of glucose, urea, creatinine, and total proteins.

ANOVA statistical determination was used on ranks followed by All Pairwise Multiple Comparison Procedures (Student-Newman-Keuls method) was the statistical method used for this Example.

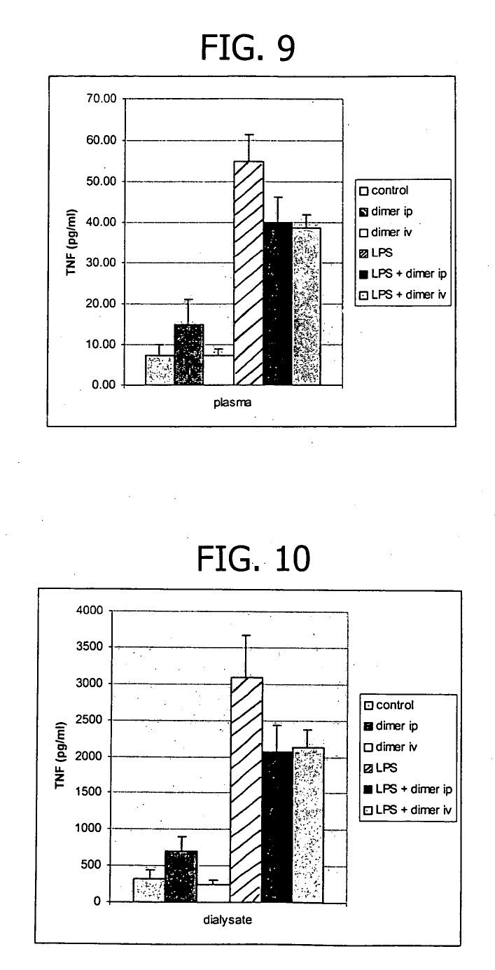

These data indicate that treatment with the KPV dimer either IV or IP caused a significant inhibition of TNF concentrations as estimated from TNF concentration in plasma and dialysate of endotoxin-treated rats (FIGS. 9 and 10). There was no significant difference in TNF concentration among the control group and groups treated with the KPV dimer IV or IP in the absence of endotoxin challenge.

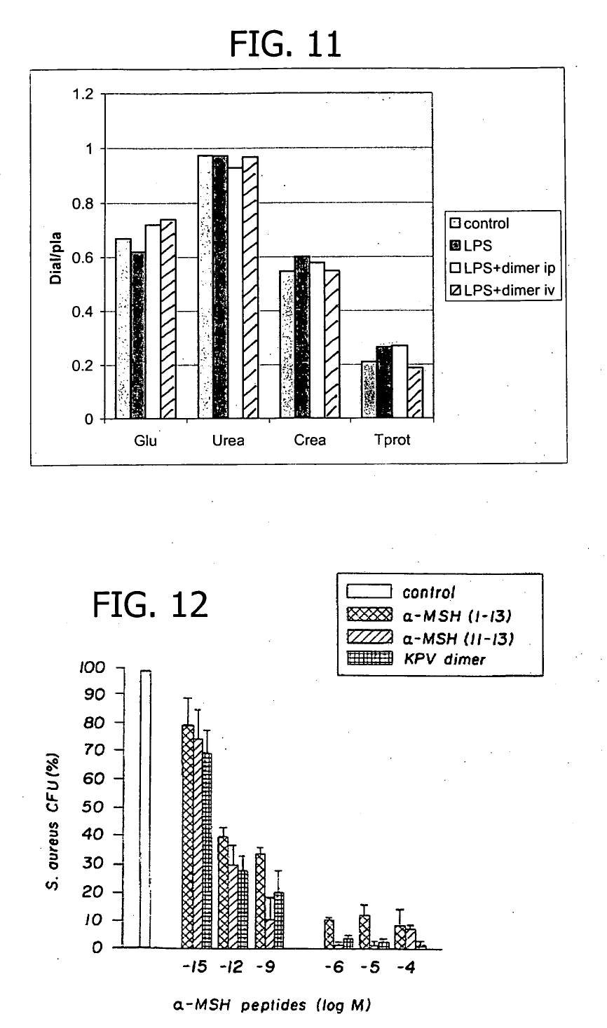

FIG. 11 illustrates that the KPV dimer (SEQ ID NO: 4) did not substantially alter peritoneal transport of small and large molecules determined by measuring dialysate/serum ratio of glucose, urea, creatinine and total plasma.

### EXAMPLE III

### Treating Bacteria and Fungi with Anti-Microbial and Anti-Inflammatory Peptides

The following microorganisms were used in the Example: Candida albicans (ATCC 10231); Enterococcus faecalis (MDR) (ATCC 51299); Escherichia coli (ATCC 11229); Pseudomonas aeruginosa (ATCC 9027); Staphylococcus aureus (ATCC 6538); Staphylococcus epidermidis (ATCC 12228); and Streptococcus pyogenes Group A (ATCC 19615). These organisms were subcultured onto blood agar plates and incubated for 16-24 hours at 35±2° C. An inoculated needle or loop was touched to each of four or five well isolated colonies of the same morphological type, and the inoculum inoculated into 5 mL of MHB.

The broth cultures were then allowed to incubate at 35±2° C. until turbidity appeared (usually 2 to 5 hours). The turbidity of actively growing broth cultures was then adjusted with saline or broth to obtain a turbidity visually comparable to that of a 0.5 McFarland Standard prepared by adding 0.5 mL of 0.048 M BaCl₂ (1.75% [wt/vol] BaCl₂ H₂O) to 99.5 mL of 0.36 N H₂SO₄ (1%, vol/vol). This turbidity is half the density of a McFarland number one standard and is often referred to as McFarland 0.5 standard (approx. 1.5×10⁸ CFU/mL). This turbidity standard was agitated on a vortex mixer immediately before use. Sealed tubes were stored in the dark at room temperature and replaced if the inocula are shown (by count) to be aberrant.

The broth culture was further diluted to 1:200 in MHB broth (to obtain a final concentration of 10⁵ to 10⁶ CFU/mL). To determine the initial concentration of the organism in the above solution, serial dilutions were performed 1:10 in MHB four times to achieve a final dilution of 10⁻⁴. Aliquots 1.0 mL from each of the dilutions were placed in duplicate into sterile petri dishes. Approximately 15 to 20 mL of tempered (45±2° C.) MCTA agar media was added. The plates were incubated overnight at 35±2° C. for 16-20 hours. Following the incubation period, the colonies were counted. The initial concentration of the organisms contained 10⁵ to 10⁶ CFU/mL.

The formula weight for each of the four anti-microbial and anti-inflammatory peptides are shown in Table 1.

TABLE 1

| Description | Formula Weight |
|---|---|
| KPV (SEQ ID NO: 1) | 384 |
| KPV dimer (SEQ ID NO: 4) | 971.26 |
| NDP (SEQ ID NO: 7) | 1646.8 |
| Phe7 (SEQ ID NO: 5) | 1126.4 |

Anti-microbial and anti-inflammatory peptides were prepared to result in concentration of 2.0×10⁻³ M to 2.0×10⁻¹¹ M solutions. These dilutions were developed according to the procedure: for each drug substance label 9 flasks (10 ml Volumetric) 1 through 9; for each sample add 3 mL of deionized (“DI”) water to a 10 mL volumetric flask; then transfer the amount of anti-microbial and anti-inflammatory peptides as specified below in Table 2; record the actual amount transferred in the laboratory notebook; and add the remaining amount of DI water to bring up to volume of 10 mL.

TABLE 2

| | mg of drug |
|---|---|
| Internal | substance to be |
| Accession # | added |
| KPV (SEQ ID | 7.68 |
| NO: 1) | |
| KPV dimer | 19.43 |
| (SEQ ID NO: 4) | |
| NDP (SEQ ID | 32.94 |
| NO: 7) | |
| Phe7 (SEQ ID | 22.53 |
| NO: 5) | |

100 μL of the anti-microbial and anti-inflammatory peptide solution from each of the flasks was then added to the correspondingly labeled well; to well number 10 add 100 μL sterile DI Water; and to all ten wells add 100 μL of inoculum created previously.

This resulted in the following M drug concentration in each well, which is shown in Table 3.

TABLE 3

| Well 1 | Well 2 | Well 3 | Well 4 | Well 5 | Well 6 | Well 7 | Well 8 | Well 9 | Well 10 |
|---|---|---|---|---|---|---|---|---|---|
| 10⁻³ | 10⁻⁴ | 10⁻⁵ | 10⁻⁶ | 10⁻⁷ | 10⁻⁸ | 10⁻⁹ | 10⁻¹⁰ | 10⁻¹¹ | Growth |
| | | | | | | | | | control |

The procedure was repeated for each of the seven microorganisms tested. The inoculated tubes were incubated at 35±2° C. for 16-20 hours or as long as necessary for the growth control, well, #10, to exhibit turbidity.

Each well was tested for turbidity, as an indicator of growth.

The results indicated that all the anti-microbial and anti-inflammatory peptides are very effective against Candida albicans; growth of this organism was completely inhibited by concentrations of these peptides (Table 4). Both KPV and Phe7 were likewise effective against Enterococcus faecalis. Of particular interest to the example is that the Phe7 molecule was very effective against Staphylococcus epidermidis, an organism known to infect catheter wound sites. Both KPV and the Phe7 molecule were effective against Streptococcus pyogenes. Table 4 includes only those anti-microbial and anti-inflammatory peptides that completely inhibited growth. Each anti-microbial and anti-inflammatory peptide in the example showed marked cidal activity against the selected microbe. Under the conditions used in these experiments, none of the peptides completely inhibited the growth of Escherichia coli, Pseudomonas aeruginosa, or Staphylococcus aureus.

Table 4 shows the minimal effective concentrations of tested peptides that completely inhibited growth of yeast and bacteria.

TABLE 4

| | KPV | KPV dimer | NDP | Phe7 |
|---|---|---|---|---|
| Candida Albicans | 10⁻³ | 10⁻³ | 10⁻³ | 10⁻³ |
| Enterococcus | 10⁻³ | | | 10⁻³ |
| Faecalis | | | | |
| Escherichia Coli | | | | |
| Pseudomonas | | | | |
| Aeruginosa | | | | |
| Staphylococcus | | | | |
| Aureus | | | | |
| Staphylococcus | | | | 10⁻⁴ |
| Epidermidis | | | | |
| Streptococcus | 10⁻³ | | | 10⁻⁴ |
| Pyogenes | | | | |

### EXAMPLE IV

### Use of Anti-Microbial and Anti-inflammatory Peptides to Decrease Viability of Staphylococcus Aureus

Staphylococcus aureus (ATCC29213) was obtained from the collection of the Department of Microbiology, Ospedale Maggiore di Milano. S. aureus (1×10⁶/ml in HBSS) was incubated in the presence or absence of α-MSH [1-13] (SEQ ID NO: 6), α-MSH [11-13] (SEQ. ID NO. 1) or the KPV dimer (SEQ ID NO: 4) at concentrations in the range of 10⁻¹⁵ to 10⁻⁴M for 2 hours at 37° C. S. aureus were then washed in cold distilled water and diluted with HBSS to a concentration of 100 organisms/ml. One ml aliquots were dispensed on blood agar plates and incubated for 24 hours at 37° C. Organism viability was estimated from the number of colony forming units. As shown in FIG. 12, α-MSH [1-13] (SEQ. ID NO. 4) and α-MSH [11-13] (KPV) (SEQ. ID NO.1) inhibited S. aureus colony formation. The KPV dimer (SEQ ID NO: 4) also inhibited S. aureus colony formation. The inhibitory effect occurred over a wide range of concentrations and was statistically significant (p<0.01) with peptide concentrations of 10⁻¹² to 10⁻⁴M.

### EXAMPLE V

### The Anti-Microbial and Anti-Inflammatory Peptides Decrease the Viability of Urokinase-Induced Growth-Enhanced Staphylococcus Aureus

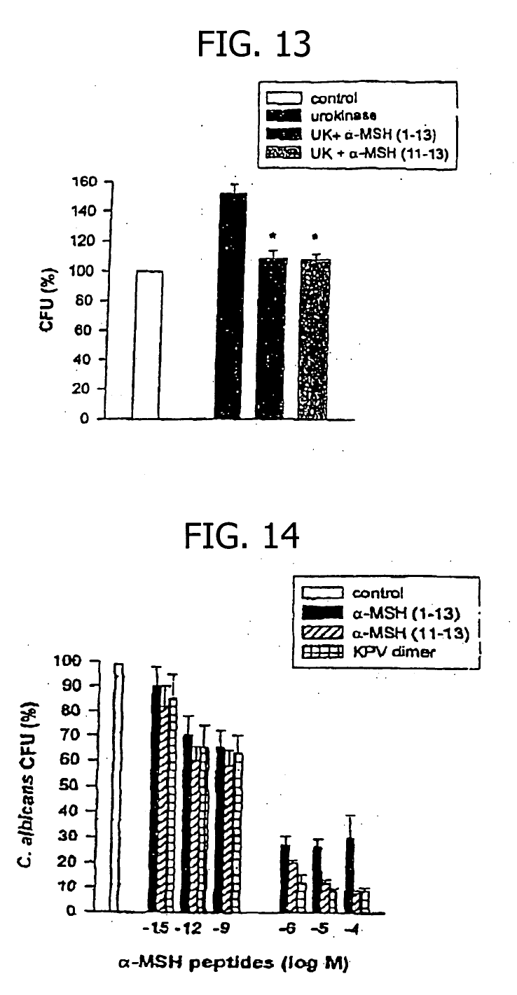

In this experiment, the influence of α-MSH on urokinase-induced growth-enhancement is determined. (Hart, D. A.; Loule, T.; Krulikl, W.; Reno, C., Staphylococcus Aureus Strains Differ in Their in Vitro Responsiveness to Human Urokinase: Evidence that Methicillin-Resistant Strains are Predominantly Nonresponsive to the Growth-Enhancing Effects of Urokinase, Can. J. Microbiol. 42, 1024-31 (1966).) S. aureus (10⁵/100 ml) were incubated for four hours at 37° C. with recombinant human urokinase 500 U (Lepetit, Milan, Italy) in a shaking water bath, in the presence or absence of α-MSH [1-13] (SEQ ID NO: 6) or α-MSH [11-13] (SEQ. ID NO.1) at 10⁻⁶ M. Appropriate dilutions of S. aureus were dispensed on agar plates and colonies counted after 24 hours incubation at 37° C. As shown in FIG. 13, the treatment with urokinase increased S. aureus colony formation and addition of α-MSH (SEQ ID NO: 6) or KPV (SEQ ID NO: 1) at concentrations of 10⁻⁶M significantly inhibited the enhancing effect of urokinase.

### EXAMPLE VI

### The Peptides Decrease the Viability of Candida albicans

C. albicans (clinical isolate) were obtained from the collection of the Department of Microbiology, Ospedale Maggiore di Milano. C. albicans were maintained on Sabouraud's agar slants and periodically transferred to Sabouraud's agar plates and incubated for 48 hours at 28° C. To prepare stationary growth phase yeast, a colony was taken from the agar plate and transferred into 30 ml Sabouraud-dextrose broth and incubated for 72 hours at 32° C. Cells were centrifuged at 100×g for 10 minutes and the pellet was washed twice with distilled water. Cells were counted and suspended in Hank's balanced salt solution (“HBSS”) to the desired concentration. Viability, determined by the exclusion of 0.01% methylene blue, remained >98%. C. albicans (I×10⁶/ml in HBSS) was incubated in the presence or absence of α-MSH [1-13], KPV, (SEQ. ID NO.1) or the (“KPV dimer”) at concentrations in the range of 10⁻¹⁵ to 10⁻⁴M for 2 hours at 37° C. Cells were then washed in cold distilled water and diluted with HBSS to a concentration of 100 organisms/ml. One ml aliquots were dispensed on blood agar plates and incubated for 48 hours at 37° C. Organism viability was estimated from the number of colonies formed. As shown in FIG. 14, C. albicans the KPV dimer (SEQ ID NO: 4) also inhibited C. albicans colony formation. Concentrations of all three peptides from 10⁻¹³ to 10⁻⁴M had significant inhibitory influences on CFU (p<0.01 vs. control).

### EXAMPLE VII

### Potency of Among the Peptides in Reducing C. albicans Viability in Comparison with Fluconazole and ACTH

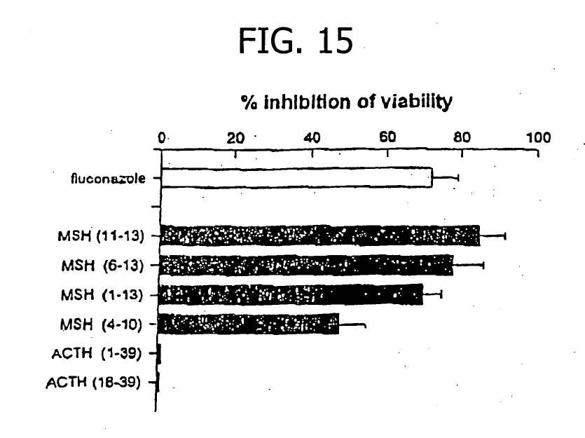

Fluconazole is a well established antifungal agent. The potency of the peptides in reducing C. albicans viability is studied in comparison with fluconazole and ACTH using similar procedures as in the examples above. The peptides and fluconazole were tested in concentrations of 10⁻⁶ M. There were at least six replicates for each concentration of peptide. As shown in FIG. 15, α-MSH [11-13] (KPV), (SEQ ID NO: 1) α-MSH [6-13] (SEQ ID NO: 2), and α-MSH [1-13] (SEQ ID NO: 6) were the most effective. Their inhibitory activity was similar to that of fluconazole. The “core” α-MSH sequence, α-MSH [4-10] (SEQ ID NO: 14) caused approximately 50% inhibition of CFU. Although this inhibitory effect was substantial (p<0.01. vs. control), it was significantly less than that caused by α-MSH fragments bearing the KPV signal sequence, i.e., α-MSH [6-13] (SEQ ID NO: 3) and α-MSH [11-13] (SEQ ID NO: 1) (p<0.01), or the parent molecule α-MSH [1-13] (SEQ ID NO: 6) (p<0.05). ACTH (1-39) (SEQ ID NO: 8) and the ACTH fragment (18-39) (SEQ ID NO: 15) did not reduce C. albicans viability (FIG. 15). Even higher concentrations of these ACTH peptides (up to 10⁻⁴ M) were likewise ineffective in reducing C. albicans CFU (results not shown in the figures).

### EXAMPLE VIII

### In vivo Tests of the KPV Dimer (SEQ ID NO: 4) Against S. Aureus and S. Epidermidis Infection at Peritoneal Catheter Exit Sites

Thirty-six white New Zealand rabbits (18 male and 18 female) divided into 6 groups: 3 male and 3 female each. A standard peritoneal dialysis catheter was implanted in each animal.

The following groups were included: Group 1 (G1)—S. aureus+no treatment; Group 2 (G2)—S. aureous+Vehicle Gel; Group 3 (G3)—S. aureus+the KPV dimer (SEQ ID NO: 4) Gel (0.1%); Group 4 (G4)—S. Epidermidis+no treatment; Group 5 (G5)—S. epidermidis+Vehicle Gel; and Group 6 (G6)—S. epidermidis+the KPV dimer (SEQ ID NO: 4) Gel (0.1%).

Catheters were implanted into the visceral cavity in the oblicuus externus abdominis area of all 36 rabbits. The rabbits were anesthetized (isofluorane inhalation), the abdominal skin was clipped, and the area sterilized using betadyne and alcohol. A midline 2 cm incision was made in the skin and abdominal muscle wall and a five inch length of catheter tubing was inserted through this incision into the peritoneal space. The tubing was secured to the abdominal muscle wall with 2-0 suture and the end passed through the abdominal wall to make an external port. A recovery period of three days was allowed before the study began on day 0 (see Table 4). S. aureus bacteria was administered to groups 1-3 and S epidermidis bacteria to groups 4-6 at the skin surrounding the catheter after KPV dimer (0.1 % in standard topical gel) or control gel application. Bacterial inoculation of the catheter site was performed by applying 1 mL isotonic saline containing the appropriate bacteria (Staphylococcus aureus or Staphylococcus epidermidis) to the surface adjacent to the catheter and evenly spreading the suspension around the catheter using sterile cotton swabs. The bacterial concentration used to inoculate the catheter sites was 1×10⁸ bacteria per mL. This amount was confirmed by serial dilution of the saline, plating an aliquot (100 μL) and counting the colonies following a forty-eight hour incubation period. The KPV dimer (SEQ ID NO: 4) or control gel was applied twice daily for six days. Swabs were taken from the local skin surrounding the catheter exit in each rabbit of all 6 groups on days 0, 2, 4, and 6. These swabs were used to inoculate blood agar plates to determine if the KPV dimer (SEQ ID NO: 4) inhibited growth of the bacteria.

TABLE 5
Test groups and animal identification number assigned to each group.

| | Male | Female |
|---|---|---|
| Staphylococcus aureus - | 4 | 1 |
| no treatment | 5 | 2 |
| | 6 | 3 |
| Staphylococcus aureus - | 7 | 10 |
| Zengen vehicle treatment | 8 | 11 |
| | 9 | 12 |
| Staphylococcus aureus - | 13 | 16 |
| Zengen the KPV dimer | 14 | 17 |
| (SEQ ID NO: 4) | 15 | 18 |
| treatment | | |
| Staphylococcus | 19 | 22 |
| epidermidis - no | 20 | 23 |
| treatment | 21 | 24 |
| Staphylococcus | 25 | 28 |
| epidermidis - Zengen | 26 | 29 |
| vehicle treatment | 27 | 30 |
| Staphylococcus | 31 | 34 |
| epidermidis - Zengen the | 32 | 35 |
| KPV dimer (SEQ ID NO: | 33 | 36 |
| 4) treatment. | | |

To be certain of direct influences on these two organisms, preliminary tests were performed on the influence of the KPV dimer (SEQ ID NO: 4) on growth of these organisms in vitro. The following procedures were used: 100 microliters of S. aureus and 100 microliters of S. epidermidis were pipetted on two blood agar plates each and incubated at 37 degrees Celsius for 24 hours; following the 24 hour incubation, the plates were removed from the incubator, and 500 microliters of the KPV dimer (SEQ ID NO: 4) compound or vehicle were applied by pipette to the bacterial cultures; the blood agar plates were replaced in the incubator at 37 degrees Celsius for 24 hours; simultaneously, on two other plates, the KPV dimer (SEQ ID NO: 4) compound or gel vehicle were applied; after the application, S. aureus or S. epidermidis 10⁸ were pipetted onto the plates; plates were incubated for 24 hours; and the plates were removed for counting and the bacterial colonies were observed microscopically.

Bacterial growth was found on all plates, with the exception of the area where the KPV dimer (SEQ ID NO: 4) and, to a lesser extent, control gel were placed. Examination of the area of inhibition under high power showed a completely clear zone surrounding the sites of the KPV dimer (SEQ ID NO: 4) application. These observations indicate that the KPV dimer (SEQ ID NO: 4) inhibits growth of both of these bacterial forms. An unexpected result was that the vehicle alone had some inhibitory effect. In practice, this could well benefit the patient using the gel to control microbial invasion.

There was marked inhibition of bacterial growth of bacteria taken from skin sites around catheters in each the KPV dimer (SEQ ID NO: 4) test group. Visual inspection of the Staphylococcus aureus and Staphylococcus epidermidis combined data indicated that treatment with the KPV dimer (SEQ ID NO: 4) reduces bacterial colony formation for both organisms.

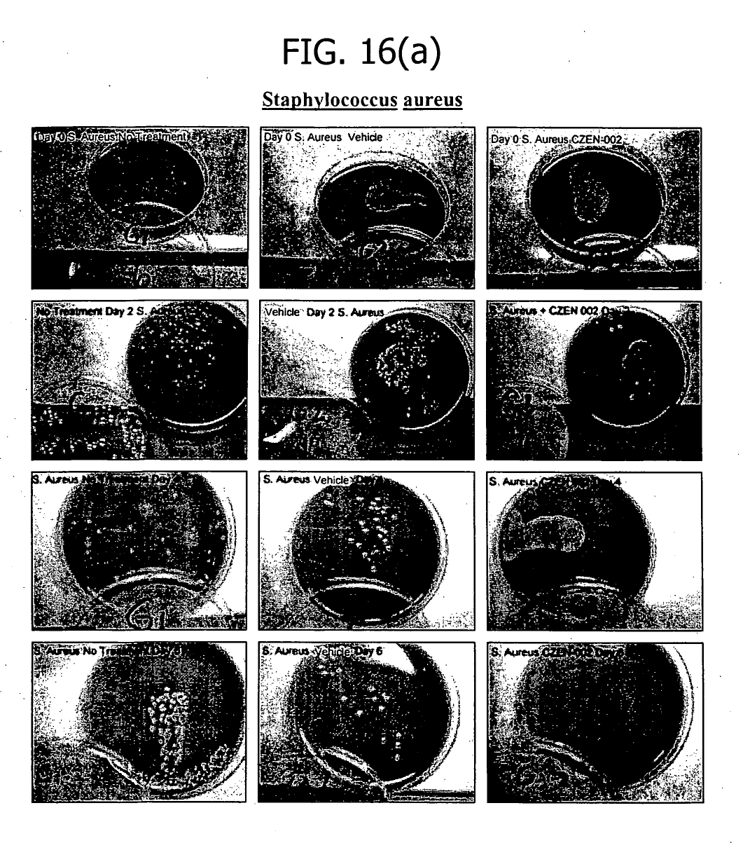

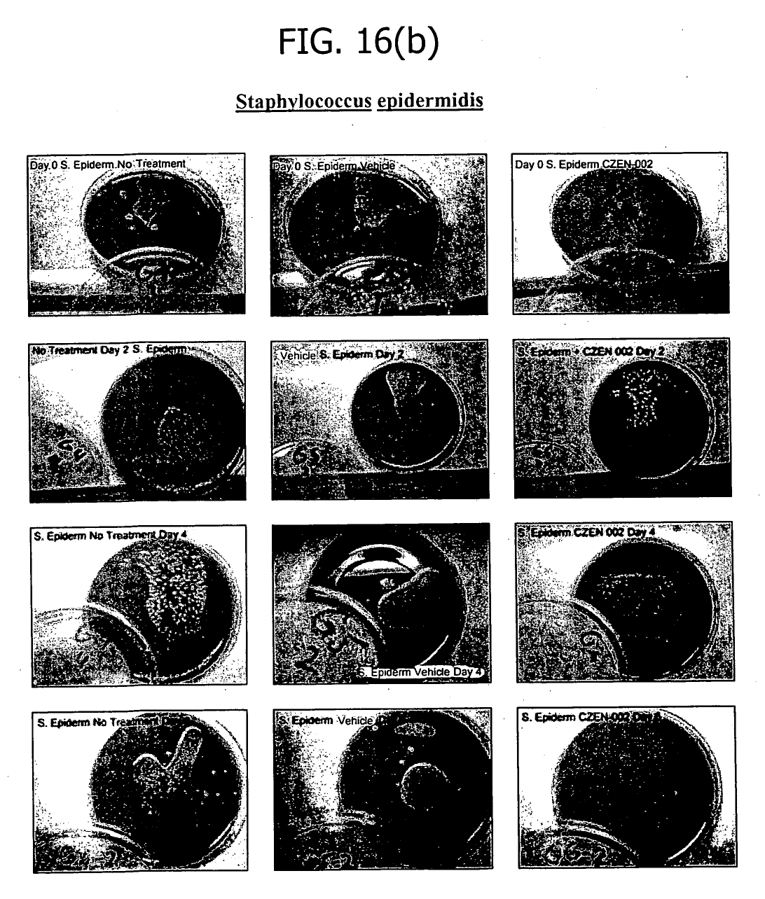

Application of the KPV dimer (SEQ ID NO: 4) gel prior to application of S. aureus and S. epidermidis bacteria in G2-3 and G5-6 (G2-G6) revealed considerable inhibition of the growth of the subsequent bacterial colonies (FIGS. 16A and B). By day 6, there were no bacterial colonies in the S. epidermidis group treated with the KPV dimer (SEQ ID NO: 4) gel nor any bacterial colony formation in the S. aureus groups treated with the same compound. The group treated with the control gel showed slight reduction in the formation of bacterial colonies.

Visual inspection of the catheter sites in rabbits given Staphylococcus aureus or S. epidermidis showed a trend toward more rapid healing in addition to the reduction of bacterial colonization. This was observed in animals of both sexes and may be a secondary benefit of the treatment.

### EXAMPLE IX

### The KPV Dimer (SEQ ID NO: 4) in a Locking Solution: Control of Infection in vitro and in vivo

In this example it is shown that the KPV dimer (SEQ ID NO: 4) in a saline locking solution is useful in the inhibition of microbes that commonly infect peritoneal catheters in humans. Tests were performed in vitro using locking fluid placed in standard catheters and held in an incubator. Additional tests were performed in vivo in rabbits bearing peritoneal catheters. In both studies, the catheters were seeded with S. epidermidis or S. aureus.

Standard peritoneal catheters were inoculated with Staphylococcus aureus or S. epidermidis, and then assigned to Groups A and B. For Group A: the KPV dimer (SEQ ID NO: 4): subgroup a. Staphylococcus aureus (2 catheters): subgroup b. Staphylococcus epidermidis (2 catheters); and subgroup c. No bacterial infection—control group (2 catheters). For Group B saline: subgroup a. Staphylococcus aureus (2 catheters); subgroup b. Staphylococcus epidermidis (2 catheters); and subgroup c. No bacterial infection—control group (two catheters).

Each Group A catheter was flushed with 0.1 % of the KPV dimer (SEQ ID NO: 4) in normal saline solution, bacteria (10⁸ CFU) were applied to subgroups a. and b. and all catheters were incubated at 37 degrees C. for 12 hours. After this incubation, 100 microliters of fluid was withdrawn from each catheter. The fluid from each catheter was then used to inoculate individual blood agar plates. The blood agar plates were incubated for 24 hours for determination of bacterial growth. In Group B the procedures were the same except that the catheters were flushed with saline alone.

The effects of the KPV dimer (SEQ ID NO: 4) on bacterial growth in peritoneal catheters in vitro can be summarized for Group A as subgroup a) the blood agar plates treated with the KPV dimer (SEQ ID NO: 4) showed no growth of the inoculated Staphylococcus aureus after 24 hour incubation; subgroup b) the blood agar plates treated with the KPV dimer (SEQ ID NO: 4) likewise showed no growth of the inoculated Staphylococcus epidermidis after 24 hour incubation; and subgroup c) the two plates treated with the KPV dimer (SEQ ID NO: 4) that had not been inoculated with bacteria showed no bacterial growth. The effects of the saline treated catheters in Group B may be summarized as: subgroup a) the two plates inoculated with Staphylococcus aureus and treated with control saline solution grew two colonies of Staphylococcus aureus during the 24 hour incubation period; subgroup b) in the plates inoculated with Staphylococcus epidermidis and treated with saline solution, one colony developed during incubation; and subgroup c) the two plates treated with saline solution and no bacterial inoculation showed no bacterial growth after incubation.

The KPV dimer (SEQ ID NO: 4) in saline locking solution inhibited the capacity of both S. aureus and S epidermidis to form colonies in a salutary culture. These results support a use of the KPV dimer (SEQ ID NO: 4) in locking solutions.

### EXAMPLE X

### In vivo Locking Solution Studies

Peritoneal catheters were implanted in New Zealand white rabbits using the techniques described above. After the recovery period, either the KPV dimer (SEQ ID NO: 4) 0.1 % in saline or saline alone was added to the catheters along with the bacteria (10⁸ CFU/ml). After 12 hours 100 microliters of the fluid in the catheters was withdrawn and cultured (24 hr) for colony counts. This example included the KPV dimer (SEQ ID NO: 4) Group and the Saline Control Group. In the KPV dimer (SEQ ID NO: 4)Group: a. Staphylococcus aureus (two rabbits) and b. Staphylococcus epidermidis (two rabbits). In the Saline Control Group: a. Staphylococcus aureus (two rabbits) and b. Staphylococcus epidermidis (two rabbits).

The following inoculations were performed: Group 1 a.: Two catheters flushed with the KPV dimer (SEQ ID NO: 4) followed by inoculation with Staphylococcus aureus 10⁸ CFU; Group 1 b.: Two catheters flushed with the KPV dimer (SEQ ID NO: 4) followed by inoculation with Staphylococcus epidermidis 10⁸ CFU; Group 2 a.: Two catheters flushed with saline solution followed by inoculation with Staphylococcus aureus 10⁸ CFU; Group 2 b.: Two catheters flushed with saline solution followed by inoculation with Staphylococcus epidermidis 10⁸ CFU

The results are summarized herein. Aliquots extracted from the two implanted catheters that were treated with the KPV dimer (SEQ ID NO: 4) locking solution and inoculated with Staphylococcus aureus were plated and incubated for 24 hours. No colonies developed in these plates. Aliquots extracted from the two implanted catheters that were treated with the KPV dimer (SEQ ID NO: 4) locking solution and inoculated with Staphylococcus epidermidis were plated and incubated for 24 hours. Seven colonies developed on these plates. Aliquots extracted from the two implanted catheters that were treated with saline solution and inoculated with Staphylococcus aureus were plated and incubated for 24 hours. Over 50 colonies of Staphylococcus aureus developed on the plates. Aaliquots extracted from the two implanted catheters that were treated with saline solution and inoculated with Staphylococcus epidermidis were plated and incubated for 24 hours. Over 100 colonies developed on these plates.

The results of the above examples are quite consistent. For the in vitro, example, both, bacteria, Staphylococcus aureus and Staphylococcus epidermidis were inhibited in vitro by the locking solution containing the KPV dimer (SEQ ID NO: 4). In the in vivo example, aliquots from the specimens treated with the KPV dimer (SEQ ID NO: 4) locking solution prior to inoculation showed complete inhibition of the Staphylococcus aureus colony formation. Aliquots from the specimens treated with the KPV dimer (SEQ ID NO: 4) locking solution prior to the bacterial inoculation showed major inhibition of the Staphylococcus epidermidis colony. Thus, both prevention and treatment options for the KPV dimer (SEQ ID NO: 4) are established.

### EXAMPLE XI

### The Peptides Inhibit HIV-p24 Expression in HIV Infected Cells

An HIV-1 infected promonocytic UI cell line was maintained in complete culture medium (RPMI 1640 supplemented with 10 mM Hepes), 2 mM L-glutamine (Sigma-Aldrich), 10% heat inactivated FCS (HyClone Laboratories, Logan, Utah, USA), penicillin at 100 units/mL and streptomycin at 100 (g/mL (Gibco Laboratories, Grand Island, N.Y.) in log phase of growth. Before use, cells were washed three times with HBSS (Gibco) to remove extracellular virus. Cells were plated onto 24-well flat-bottomed plates at a concentration of 2×10^{6 /}mL (final volume 1 mL) with medium plus TNF-α (10 ng/mL (R&D Systems, Oxford, England, UK) in the presence or absence of α-MSH peptides in concentrations from 10⁻¹³ to 10⁻⁴ M. Supernatants were removed by centrifugation after 48 hr incubation at 37° C. in 5% CO₂, and tested for HIV-p24 release. p24 antigen releases (Cellular Products Inc., Buffalo, N.Y., USA) were determined using commercial ELISA kits. In all experiments each condition was tested in triplicate.

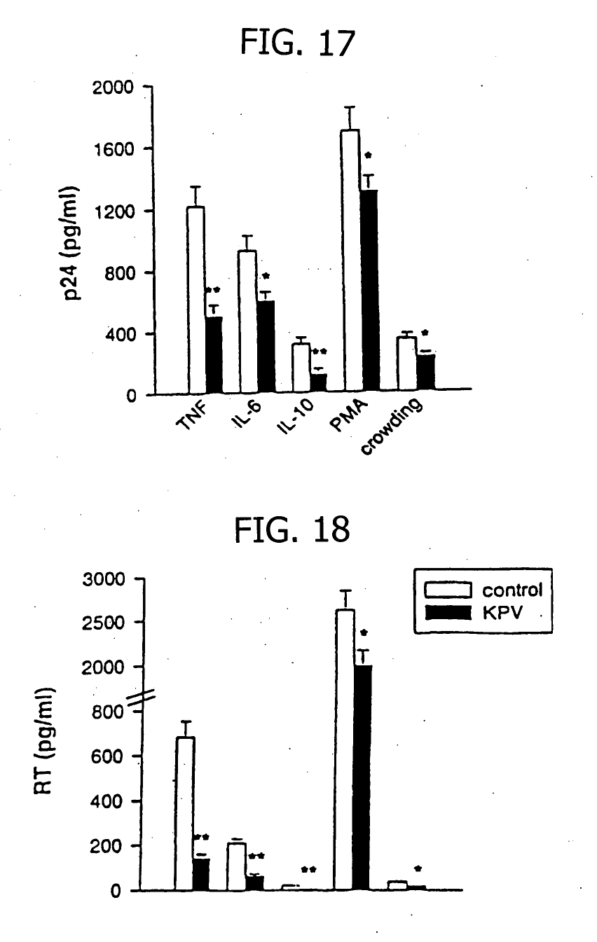

HIV-p24 is a capside HIV structure protein. The level of HIV-p24 reflects HIV infection and HIV viral amount. As shown in FIG. 17, α-MSH (SEQ ID NO: 6) and the tripeptide KPV (SEQ. ID NO.1) significantly inhibited p24 release from TNF-α stimulated UI cells. Inhibitory effects of α-MSH peptides occurred over a broad range of peptide concentrations including picomolar concentrations that occur in human plasma. Greater concentrations caused more pronounced HIV inhibition, with the most effective concentration for both peptides being 10⁻⁵ M. In this concentration, α-MSH (SEQ ID NO: 6) and KPV (SEQ. ID NO.1) caused 52.7% and 56.0% inhibition of p24 release, respectively.

### EXAMPLE XII

### The Peptides Inhibit HIV-p24 and Reverse Transcriptase Expression in HIV Infected Cells Stimulated by TNF-α, IL-6, IL-10, and PMA

HIV-1 infected promonocytic UI cells were plated onto 24-well flat-bottomed plates at a concentration of 2×10 /mL (final volume 1 mL) with medium alone or TNF-α (10 ng/mL), IL-6 (20 ng/mL), IL-10 (20 ng /mL (R&D Systems) or PMA (I ng/mL) (Sigma-Aldrich Chemicals, St. Louis, Mo., USA) in the presence or absence of KPV (SEQ. ID NO.1) in concentrations of 10⁻⁵ M. Supernatants were removed by centrifugation after 48 hr incubation at 37° C. in 5% CO₂, and tested for HIV-p24 release and reverse transcriptase release. In crowding experiments, UI cells were seeded at the density of 2×10⁵ mL and maintained in culture at 37° C. in 5% CO₂ without change of medium for 7 days. KPV (SEQ. ID NO. 1), in concentrations of 10⁻⁵M, were added on day 1. p24 antigen releases (Cellular Products Inc., Buffalo, N.Y., USA) and reverse transcriptase (ELISA Retrosys RT assay, Innovagen, Lund, Sweden) were determined using commercial ELISA kits. In all experiments, each condition was tested in triplicate. As shown in FIG. 18, KPV (SEQ. ID NO.1) significantly inhibited p24 and RT release from UI cells induced by IL-6, IL-10, PMA, and in crowding conditions.

### EXAMPLE XIII

### The Peptides Inhibit HIV Transcription

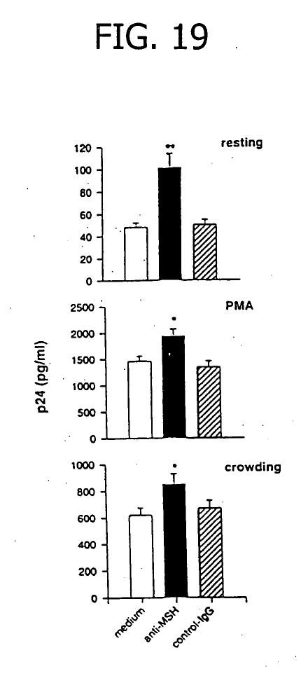

To determine the influence of KPV (SEQ. ID NO. 1) on HIV transcription, 2×10⁶ UI cells (at a density of 2×10⁵/mL in complete medium) were stimulated for 24 h with PMA (1 ng/mL) in the presence or absence of KPV (SEQ. ID NO.1) 10⁻⁵M. Total RNA was extracted by the guanidine thiocyanate phenol method using an RNA isolation kit (Tripure, Boehringer Mannheim, Indianapolis, Ind.), following the manufacturer's instructions. Ten ng of total RNA were separated by 0.8% agarose/formaldehyde gel electrophoresis and transferred onto nylon membrane. The filters were baked and hybridized for 18 hr with ³²P-labeled HIV-full length probe (kind gift of L. Turchetto and E. Vicenzi, S. Raffaele Hospital, Milan, Italy). The radiolabeling reaction was performed using a DNA labeling kit (Ready-to-go, Pharmacia Biotech, San Francisco, Calif.). Filters were washed and exposed to X-ray film for 5 days. The labeled probe was removed by washing at 80° C. in 0.1×SSC containing 0.1% sodium dodecyl sulphate and then rehybridized with (³²P-labeled glyceraldehyde-3-phosphate dehydrogenase) (GAPDH) cDNA probe. Densitometric analysis was performed using ImageMaster VDS 3.0 software (Pharmacia Biotech) and results were expressed as density units. As shown in FIG. 19, the inhibitory activity of KPV (SEQ. ID NO.1) on HIV transcription was confirmed by Northern blot analysis of HIV-RNA in PMA-stimulated UI cells. Addition of KPV (SEQ. ID NO.1) reduced by approximately 50% both spliced and unspliced HIV-1 RNA in PMA-stimulated UI cells.

From the foregoing, it will be appreciated that specific embodiments of the invention have been described herein for purposes of illustration, but that various modifications may be made without deviating from the spirit and scope of the invention. Accordingly, the invention is not limited except as by the appended claims. The preceding Examples are intended only as examples and are not intended to limit the invention. It is understood that modifying the examples above does not depart from the spirit of the invention. It is further understood that the each example may be applied on its own or in combination with other examples.

1. A wound care gel comprising a therapeutically effective amount of a peptide with a C-terminus sequence KPV in a gel comprising carbopol, propylparaben, methylparaben, propylene glycol, EDTA, 2M NaOH solution, and water.

2. The gel of claim 1 wherein the peptide comprises 3-13 amino acids.

3. The gel of claim 2 wherein the peptide may be selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

4. The gel of claim 3 wherein the peptide is SEQ ID NO: 4.

5. The gel of claim 4 wherein the peptide is a dimer.

6. The gel of claim 1 wherein the peptide is at least about 0.01 to 0.9% in the gel.

7. The gel of claim 1 wherein the peptide is at least about 0.1% in the gel.

8. A dialysate comprising: a therapeutically effective amount of a peptide with a C-terminus sequence KPV; glucose; sodium chloride; sodium lactate; magnesium chloride; calcium chloride; and glucose monohydrate.

9. The dialysate of claim 8 wherein the peptide may be selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

10. The dialysate of claim 9 wherein the peptide comprises about 3-13 amino acids.

11. The dyalisate of claim 10 wherein the peptide is a SEQ ID NO: 4.

12. The dyalisate of claim 11 wherein the peptide is a dimer.

13. The dialysate of claim 8 wherein the glucose is 1 to 5 percen; the sodium chloride is 3 to 6 grams; the magnesium chloride is 0.005 to 0.12 grams; and the glucose monohydrate is 25 to 50 grams.

14. The dialysate of claim 8 wherein the therapeutically effective amount is selected from the group consisting of within the range of at least about 1×10⁻³ to 1×10⁻⁹M and within the range of at least about 2×10⁻³ to 2×10⁻¹¹ M.

15. A locking solution comprising: an anticoagulant; and a therapeutically effective amount of a peptide with a C-terminus sequence KPV.

16. The locking solution of claim 15 wherein the peptide comprises 3-13 amino acids.

17. The locking solution of claim 16 wherein the peptide may be selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

18. The locking solution of claim 17 wherein the peptide is a SEQ ID NO: 4.

19. The locking solution of claim 18 wherein the peptide is a dimer.

20. The locking solution of claim 15 further comprising a solvent.

21. The locking solution of claim 15 wherein the anticoagulant is selected from the group consisting of heparin, danaparoid and lepirudin.

22. The locking solution of claim 15 wherein the solvent is selected from the group consisting of Ringers Lactate, 5% dextrose and water, saline and water.

23. The locking solution of claim 15 wherein the therapeutically effective amount of the peptide is selected from the group consisting of the range of at least about 1×10⁻³ to 1×10⁻⁹M and within the range of at least about 2×10⁻³ to 2×10⁻¹¹M.

24. A method of increasing diuresis in peritoneal dialysis comprising: use of a dialysate comprising a therapeutically effective amount of a peptide with a C-terminus sequence KPV.

25. The method of claim 24 wherein the peptide comprises 3-13 amino acids.

26. The method of claim 25 wherein the peptide may be selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

27. The method of claim 26 wherein the peptide is SEQ ID NO: 4.

28. The method of claim 27 wherein the peptide is a dimer.

29. The method of claim 24 wherein the therapeutically effective amount of the peptide is selected from the group consisting of within the range of at least about 1×10⁻³ to 1×10⁻⁹M and within the range of at least about 2×10⁻³ to 1×10⁻¹¹M.

30. A method of treatment of peritonitis in dialysis comprising: performance of a at least one dialysis exchange using a dyalysate comprising a at least on dose of a therapeutically effective amount of a peptide with a C-terminus sequence KPV; glucose; sodium chloride; sodium lactate; magnesium chloride; calcium chloride; and glucose monohydrate.

31. The method of claim 30 wherein the peptide comprises 3-13 amino acids.

32. The method of claim 31 wherein the peptide may be selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

33. The method of claim 32 wherein the peptide is a SEQ ID NO: 4.

34. The method of claim 33 wherein the peptide is a dimer.

35. The method of claim 30 wherein the glucose is 1 to 5 percent; the sodium chloride is 3 to 6 grams: the calcium chloride is 0.01 to 0.3 grams; the magnesium chloride is 0.005 to 0.12 grams; and the method of claim 30 wherein the glucose monohydrate is 25 to 50 grams.

36. The method of claim 30 wherein the therapeutically effective amount of the peptide is selected from the group consisting of within the range of at least about 1×10⁻³ to 1×10⁻⁹M and at least about 2×10⁻³ to 2×10⁻¹¹M

37. A method of prevention of peritonitis in dialysis comprising: performance of a at least one peritoneal dialysis exchange using a composition comprising a therapeutically effective amount of a peptide with a C-terminus sequence KPV; glucose; sodium chloride; sodium lactate; magnesium chloride; calcium chloride; and glucose monohydrate.

38. The method of claim 37 wherein the peptide comprises 3-13 amino acids.

39. The method of claim 38 wherein the peptide may be selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

40. The method of claim 39 wherein the peptide is a SEQ ID NO 4.

41. The method of claim 40 wherein the peptide is a dimer.

42. The method of claim 37 wherein the glucose is 1 to 5 percent; the sodium chloride is 3 to 6 grams; the calcium chloride is 0.01 to 0.3 grams; the magnesium chloride is 0.005 to 0.12 grams; the glucose monohydrate is 25 to 50 grams.

43. The method of claim 37 wherein the therapeutically effective amount of the peptide is selected from the group consisting of within the range of at least about 1×10⁻³ to 1×10⁻⁹M and at least about 2×10⁻³ to 2×10⁻¹¹M.

44. A kit for use in dyalysis dialysis comprising: a container containing a therapeutically effective amount of with a C-terminus sequence KPV; a container containing a solvent for dissolving the peptide; and instructions for use of the kit.

45. The kit of claim 44 further comprising an applicator selected from the group consisting of spatulas, cotton-tip applicators, syringes, spray bottles, moist towels, droppers.

46. The kit of claim 44 wherein the peptide comprises 3-13 amino acids.

47. The kit of claim 46 wherein the peptide may be selected from the group consisting of SEQ ID NO: 1, SEQ ID NO: 2, SEQ ID NO: 3, SEQ ID NO: 4, SEQ ID NO: 5, SEQ ID NO: 6, and SEQ ID NO: 7.

48. The kit of claim 47 wherein the peptide is a SEQ ID No 4.

49. The kit of claim 48 wherein the peptide is a dimer.

50. The kit of claim 44 wherein the peptide is supplied in a range selected from the group consisting of within the range of at least about 1×10⁻³ to 1 ×10⁻⁹M and at least about 2×10⁻³ to 2×10⁻¹¹M

51. The kit of claim 44 wherein the solvent is selected from the group consisting of de-ionized water, sterile saline, sterile water, Ringer's lactate, Dextrose 5% and water and combinations thereof.

52. A peptide having the sequence SYSMEHdFRWGKPV (SEQ ID NO: 5),

53. The peptide of claim 52 wherein the peptide is a dimer.


SEQUENCE LISTING (16 sequences)

SEQ ID NO: 1 - protein, 3 aa, Artificial
  Synthesized peptide
KPV

SEQ ID NO: 2 - protein, 8 aa, Homo sapiens
HFRWGKPV

SEQ ID NO: 3 - protein, 8 aa, Artificial
  Synthesized peptide
HXRWGKPV

SEQ ID NO: 4 - protein, 4 aa, Artificial
  Synthesized peptide
CKPV

SEQ ID NO: 5 - protein, 13 aa, Artificial
  Synthesized peptide
SYSMEHFRWGKPV

SEQ ID NO: 6 - protein, 13 aa, Homo Sapiens
SYSMEHFRWGKPV

SEQ ID NO: 7 - protein, 13 aa, Artificial
  Synthesized peptide
SYSXEHFRWGKPV

SEQ ID NO: 8 - protein, 39 aa, Homo sapiens
SYSMEHFRWGKPVGKKRRPVKVYPAKEDDEASEAFPLEF

SEQ ID NO: 9 - protein, 7 aa, Homo sapiens
MEHFRWG

SEQ ID NO: 10 - protein, 4 aa, Artificial
  Synthesized peptide
CKPV

SEQ ID NO: 11 - protein, 4 aa, Artificial
  Synthesized peptide
CKPV

SEQ ID NO: 12 - protein, 4 aa, Artificial
  Synthesized peptide
CKPV

SEQ ID NO: 13 - protein, 4 aa, Homo sapiens
HFRW

SEQ ID NO: 14 - protein, 6 aa, Homo sapiens
EHFRWG

SEQ ID NO: 15 - protein, 21 aa, Homo sapiens
PVKVYPAKEDDEASEAFPLEF

SEQ ID NO: 16 - protein, 14 aa, Artificial
  Synthesized peptide
CSYSMEHFRWGKPV